(19) United States
(12) Patent Application Publication
(10) Pub. No.: US 2020/0065970 A1
(43) Pub. Date: Feb. 27, 2020

(54) IMAGE PROCESSING APPARATUS AND STORAGE MEDIUM

(71) Applicant: OLYMPUS CORPORATION, Tokyo (JP)

(72) Inventor: Yasuko SONODA, Tokyo (JP)

(73) Assignee: OLYMPUS CORPORATION, Tokyo (JP)

(21) Appl. No.: 16/672,262

(22) Filed: Nov. 1, 2019

Related U.S. Application Data
(63) Continuation of application No. PCT/JP2017/017235, filed on May 2, 2017.

(51) Int. Cl. G06T 7/11; G06T 11/00; G06T 7/70; A61B 1/00

(52) U.S. Cl. CPC G06T 7/11; G06T 11/001; G06T 2207/30032; A61B 1/0005; G06T 7/70

(57) ABSTRACT

An image processing apparatus includes a processor that detects regions of interest for each of plural of observation images acquired by performing image pickup of an object, sequentially records the plurality of observation images as record images in either one of a first period from a first detection start at which a first region of interest starts to be detected to a first detection cessation at which detection of the first region of interest ceases and a second period from the first detection start to a second detection cessation at which detection of a second region of interest ceases, calculates a display timing at which the plurality of record images start to be reproduced, and performs processing for displaying at least one of the record images on a display screen of a display apparatus while sequentially displaying the plurality of observation images on the display screen at the display timing.

### CROSS REFERENCE TO RELATED APPLICATION

This application is a continuation application of PCT/JP2017/017235 filed on May 2, 2017, the entire contents of which are incorporated herein by this reference.

### BACKGROUND OF THE INVENTION

### 1. Field of the Invention

The present invention relates to an image processing apparatus and a storage medium.

### 2. Description of the Related Art

In a medical field, such a configuration that an image acquired by performing image pickup of an object using an endoscope is simultaneously displayed on one relatively large display region on a display screen of a display apparatus and another relatively small display region on the display screen, for example, has conventionally been known.

More specifically, Japanese Patent Application Laid-Open Publication No. 10-262923, for example, discloses a configuration for simultaneously displaying an image acquired by performing image pickup of an object using an electronic endoscope as a parent screen and a child screen respectively having different sizes on a monitor.

### SUMMARY OF THE INVENTION

An image processing apparatus according to an aspect of the present invention includes a processor. The processor performs processing for sequentially receiving a plurality of observation images acquired by performing image pickup of an object and respectively detecting regions of interest for the plurality of observation images, sequentially records the plurality of observation images as record images in either one of a first period from a first detection start at which a first region of interest starts to be detected to a first detection cessation at which detection of the first region of interest ceases and a second period from the first detection start to a second detection cessation at which detection of a second region of interest ceases when the second region of interest is detected in the first period, calculates a display timing at which the plurality of record images start to be reproduced based on a time point of at least one of the first detection start, the first detection cessation, a second detection start at which the second region of interest starts to be detected, and the second detection cessation, and performs processing for displaying at least one of the recorded record images on a display screen of a display apparatus while sequentially displaying the plurality of observation images on the display screen at the display timing.

A storage medium according to another aspect of the present invention stores a program for causing a computer to perform processing for respectively detecting regions of interest for a plurality of observation images acquired by performing image pickup of an object, processing for sequentially recording the plurality of observation images as record images in either one of a first period from a first detection start at which a first region of interest starts to be detected to a first detection cessation at which detection of the first region of interest ceases and a second period from the first detection start to a second detection cessation at which detection of a second region of interest ceases when the second region of interest is detected in the first period, and processing for calculating a display timing at which the plurality of record images start to be reproduced based on a time point of at least one of the first detection start, the first detection cessation, a second detection start at which the second region of interest starts to be detected, and the second detection cessation.

### BRIEF DESCRIPTION OF THE DRAWINGS

FIG. 1 is a diagram illustrating a configuration of a principal part of an endoscope system including an image processing apparatus according to an embodiment.

FIG. 2 is a block diagram for describing an example of a specific configuration of the image processing apparatus according to the embodiment.

FIG. 3 is a timing chart for describing an example of processing to be performed in the image processing apparatus according to the embodiment.

FIG. 4 is a diagram illustrating an example of a display image to be displayed on a display apparatus through the processing of the image processing apparatus according to the embodiment.

FIG. 5 is a diagram illustrating an example of a display image to be displayed on the display apparatus through the processing of the image processing apparatus according to the embodiment.

FIG. 6 is a diagram illustrating an example of a display image to be displayed on the display apparatus through the processing of the image processing apparatus according to the embodiment.

FIG. 7 is a diagram illustrating an example of a display image to be displayed on the display apparatus through the processing of the image processing apparatus according to the embodiment.

FIG. 8 is a timing chart for describing an example different from the example illustrated in FIG. 3 of processing to be performed in the image processing apparatus according to the embodiment.

FIG. 9 is a diagram illustrating an example of a display image to be displayed on the display apparatus through the processing of the image processing apparatus according to the embodiment.

FIG. 10 is a diagram illustrating an example of a display image to be displayed on the display apparatus through the processing of the image processing apparatus according to the embodiment.

FIG. 11 is a diagram illustrating an example of a display image to be displayed on the display apparatus through the processing of the image processing apparatus according to the embodiment.

FIG. 12 is a timing chart for describing an example different from the example illustrated in FIG. 3 of processing to be performed in the image processing apparatus according to the embodiment.

FIG. 13 is a diagram illustrating an example of a display image to be displayed on the display apparatus through the processing of the image processing apparatus according to the embodiment.

FIG. 14 is a diagram illustrating an example of a display image to be displayed on the display apparatus through the processing of the image processing apparatus according to the embodiment.

FIG. 15 is a timing chart for describing an example different from the example illustrated in FIG. 3 of processing to be performed in the image processing apparatus according to the embodiment.

FIG. 16 is a diagram illustrating an example of a display image to be displayed on the display apparatus through the processing of the image processing apparatus according to the embodiment.

FIG. 17 is a diagram illustrating an example of a display image to be displayed on the display apparatus through the processing of the image processing apparatus according to the embodiment.

FIG. 18 is a timing chart for describing an example different from the example illustrated in FIG. 3 of processing to be performed in the image processing apparatus according to the embodiment.

FIG. 19 is a diagram illustrating an example of a display image to be displayed on the display apparatus through the processing of the image processing apparatus according to the embodiment.

FIG. 20 is a timing chart for describing an example different from the example illustrated in FIG. 3 of processing to be performed in the image processing apparatus according to the embodiment.

FIG. 21 is a timing chart for describing an example different from the example illustrated in FIG. 3 of processing to be performed in the image processing apparatus according to the embodiment.

FIG. 22 is a timing chart for describing an example different from the example illustrated in FIG. 3 of processing to be performed in the image processing apparatus according to the embodiment.

FIG. 23 is a diagram illustrating an example of a display image to be displayed on the display apparatus through the processing of the image processing apparatus according to the embodiment.

FIG. 24 is a diagram illustrating an example of a display image to be displayed on the display apparatus through the processing of the image processing apparatus according to the embodiment.

FIG. 25 is a timing chart for describing an example different from the example illustrated in FIG. 3 of processing to be performed in the image processing apparatus according to the embodiment.

FIG. 26 is a timing chart for describing an example different from the example illustrated in FIG. 3 of processing to be performed in the image processing apparatus according to the embodiment.

FIG. 27 is a diagram illustrating an example of a display image to be displayed on the display apparatus through the processing of the image processing apparatus according to the embodiment.

### DETAILED DESCRIPTION OF THE PREFERRED EMBODIMENT

An embodiment of the present invention will be described below with reference to the drawings.

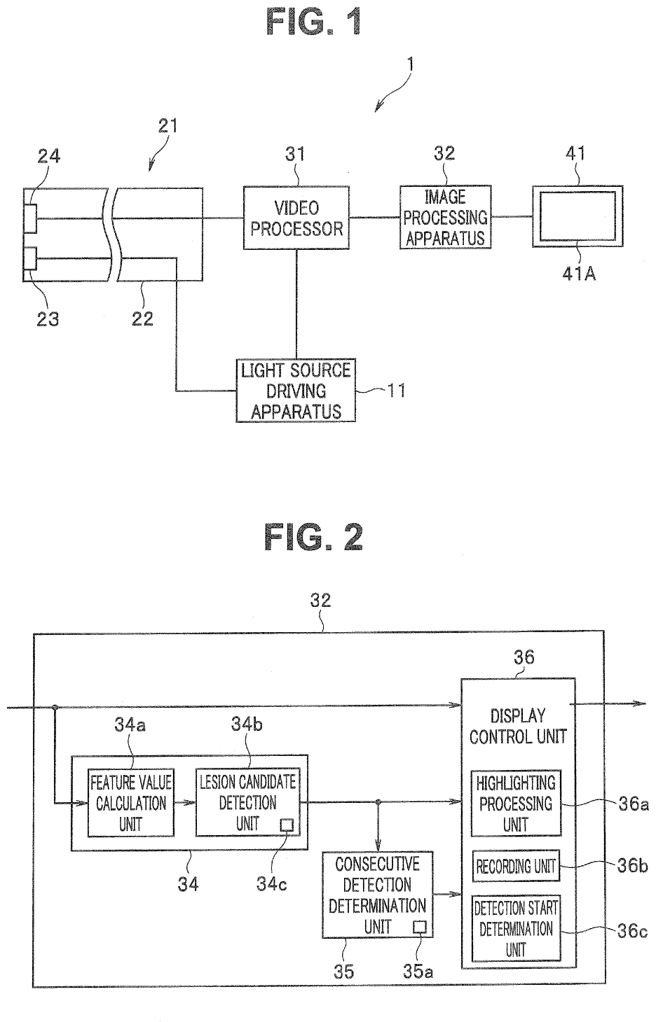

An endoscope system 1 is configured to include a light source driving apparatus 11, an endoscope 21, a video processor 31, an image processing apparatus 32, and a display apparatus 41, as illustrated in FIG. 1.

The light source driving apparatus 11 is configured to include a drive circuit, for example. The light source driving apparatus 11 is connected to the endoscope 21 and the video processor 31. The light source driving apparatus 11 is configured to generate a light source driving signal for driving a light source unit 23 in the endoscope 21 based on a light source control signal from the video processor 31 and output the generated light source driving signal to the endoscope 21.

The endoscope 21 is connected to the light source driving apparatus 11 and the video processor 31. The endoscope 21 is configured to include an elongated insertion unit 22 that can be inserted into a body cavity of an examinee A light source unit 23 and an image pickup unit 24 are provided in a distal end portion of the insertion unit 22.

The light source unit 23 is configured to include a light emitting element such as a white LED, for example. The light source unit 23 is configured to emit light in response to the light source driving signal to be outputted from the light source driving apparatus 11 to generate illumination light and emit the generated illumination light to an object such as a living tissue.

The image pickup unit 24 is configured to include an image sensor such as a color CCD or a color CMOS, for example. The image pickup unit 24 is configured to perform an operation in response to an image pickup control signal to be outputted from the video processor 31. The image pickup unit 24 is configured to receive reflected light from the object illuminated by the illumination light from the light source unit 23, pick up an image of the received reflected light to generate an image pickup signal, and output the generated image pickup signal to the video processor 31.

The video processor 31 is connected to the light source driving apparatus 11 and the endoscope 21. The video processor 31 is configured to generate a light source control signal for controlling a light emitting state of the light source unit 23 and output the generated light source control signal to the light source driving apparatus 11. The video processor 31 is configured to generate and output an image pickup control signal for controlling an image pickup operation of the image pickup unit 24. The video processor 31 is configured to subject the image pickup signal to be outputted from the endoscope 21 to predetermined processing to generate an observation image G1 of the object and sequentially output the generated observation image G1 to the image processing apparatus 32 frame by frame.

The image processing apparatus 32 is configured to include an electronic circuit such as an image processing circuit. The image processing apparatus 32 is configured to perform an operation for generating a display image based on the observation image G1 to be outputted from the video processor 31 and displaying the generated display image on the display apparatus 41. The image processing apparatus 32 is configured to include a region-of-interest detection unit 34, a consecutive detection determination unit 35, and a display control unit 36, as illustrated in FIG. 2. FIG. 2 is a block diagram for describing an example of a specific configuration of the image processing apparatus 32 according to the embodiment.

The region-of-interest detection unit 34 is configured to calculate a predetermined feature value related to each of the observation images G1 to be sequentially outputted from the video processor 31 and further detect a plurality of lesion candidate regions Ln (n=1, 2 . . . ), respectively, as regions of interest included in the observation image G1 based on the calculated predetermined feature value. In other words, the region-of-interest detection unit 34 is configured to sequentially receive the plurality of observation images G1 acquired by performing image pickup of the object using the endoscope 21 while performing processing for detecting the lesion candidate region L for each of the plurality of observation images G1. The region-of-interest detection unit 34 is configured to include a feature value calculation unit 34a and a lesion candidate detection unit 34b.

The feature value calculation unit 34a is connected to the video processor 31 and the lesion candidate detection unit 34b. The feature value calculation unit 34a is configured to calculate a predetermined feature value related to each of the observation images G1 to be sequentially outputted from the video processor 31 and output the calculated predetermined feature value to the lesion candidate detection unit 34b.

More specifically, the feature value calculation unit 34a calculates a slope value as a value representing an amount of change in luminance or an amount of change in concentration between each of pixels within one of a plurality of small regions obtained by dividing the observation image G1 in a predetermined size and each of pixels within the small region adjacent to the one small region as a feature value for each of the plurality of small regions. Note that the feature value calculation unit 34a may calculate a value different from the above-described slop value as a feature value as long as the feature value calculation unit 34a calculates a value at which the observation image G1 can be quantitatively evaluated.

The lesion candidate detection unit 34b is connected to the consecutive detection determination unit 35 and the display control unit 36. The lesion candidate detection unit 34b is configured to include a ROM 34c previously storing one or more pieces of polyp model information.

More specifically, the polyp model information stored in the ROM 34c is configured to include a feature value obtained by quantifying respective common points and/or similar points in many polyp images, for example.

The lesion candidate detection unit 34b is configured to detect a lesion candidate region Ln based on a predetermined feature value to be outputted from the feature value calculation unit 34a and plural pieces of polyp model information read from the ROM 34c, acquire lesion candidate information ILn as information representing the detected lesion candidate region Ln, and output the acquired lesion candidate information ILn to each of the consecutive detection determination unit 35 and the display control unit 36.

More specifically, the lesion candidate detection unit 34b detects, for example, when the feature value for the one small region to be outputted from the feature value calculation unit 34a and at least one of the feature values included in the plural pieces of polyp model information read from the ROM 34c match each other, the one small region as a lesion candidate region Ln. The lesion candidate detection unit 34b acquires the lesion candidate information ILn including position information and size information of the lesion candidate region Ln detected using the above-described method and outputs the acquired lesion candidate information ILn to each of the consecutive detection determination unit 35 and the display control unit 36.

Note that the position information of the lesion candidate region Ln is information representing a position of the lesion candidate region Ln within the observation image G1, and is acquired as a pixel position of the lesion candidate region Ln existing within the observation image G1, for example. The size information of the lesion candidate region Ln is information representing a size of the lesion candidate region Ln within the observation image G1, and is acquired as a number of pixels in the lesion candidate region Ln existing within the observation image G1, for example.

Note that the region-of-interest detection unit 34 may not be configured to include the feature value calculation unit 34a and the lesion candidate detection unit 34b as long as the region-of-interest detection unit 34 performs processing for detecting the lesion candidate region Ln from the observation image G1. More specifically, the region-of-interest detection unit 34 may be configured to detect the lesion candidate region Ln from the observation image G1 by performing processing for applying an image identifier that has previously acquired a function of identifying polyp images using a learning method such as deep learning to the observation image G1, for example.

The consecutive detection determination unit 35 is connected to the display control unit 36. The consecutive detection determination unit 35 is configured to include a RAM 35a capable of storing the lesion candidate information ILn preceding the lesion candidate information ILn to be outputted from the lesion candidate detection unit 34b by at least one frame.

The consecutive detection determination unit 35 is configured to determine, for example, based on first lesion candidate information to be outputted from the lesion candidate detection unit 34b and second lesion candidate information, stored in the RAM 35a, preceding the first lesion candidate information by one frame, whether or not a first lesion candidate region represented by the first lesion candidate information and a second lesion candidate region represented by the second lesion candidate information are the same lesion candidate region Ln. The consecutive detection determination unit 35 is configured to acquire, when the above-described first and second lesion candidate regions are the same lesion candidate region Ln, a determination result that the lesion candidate region Ln in the observation image G1 is consecutively detected, that is, a determination result that the lesion candidate region Ln detected by the lesion candidate detection unit 34b continues to exist within the observation image G1 and output the acquired determination result to the display control unit 36. The consecutive detection determination unit 35 is configured to acquire, when the above-described first and second lesion candidate regions are not the same lesion candidate region Ln, a determination result that the detection of the lesion candidate region Ln in the observation image G1 has ceased, that is, a determination result that the lesion candidate region Ln detected by the lesion candidate detection unit 34b has moved outward from inside the observation image G1 and output the acquired determination result to the display control unit 36.

The display control unit 36 is connected to the display apparatus 41. The display control unit 36 is configured to include a highlighting processing unit 36a, a recording unit 36b, and a detection start determination unit 36c. The display control unit 36 selects, when the region-of-interest detection unit 34 detects the plurality of lesion candidate regions Ln, the one lesion candidate region Ls among the regions. The display control unit 36 is configured to measure, when the lesion candidate information ILs is inputted from the lesion candidate detection unit 34b, a consecutive detection time period TL as a time period elapsed since the lesion candidate region Ls in the observation image G1 started to be detected based on the determination result to be outputted from the consecutive detection determination unit 35. The display control unit 36 is configured to perform processing for generating a display image using the observation images G1 to be sequentially outputted from the video processor 31 while performing processing for displaying the generated display image on a display screen 41A in the display apparatus 41.

The display control unit 36 is configured to perform, when the detection of the lesion candidate region Ln has ceased before the consecutive detection time period TL reaches a predetermined time period TH (e.g., 0.5 seconds), processing, described below, based on the determination result to be outputted from the consecutive detection determination unit 35. The display control unit 36 is configured to perform, when the lesion candidate region Ln is consecutively detected at a timing at which the consecutive detection time period TL has reached the predetermined time period TH, highlighting processing, described below, based on the determination result to be outputted from the consecutive detection determination unit 35.

The highlighting processing unit 36a is configured to start, when the lesion candidate region Ln is consecutively detected at the timing at which the consecutive detection time period TL has reached the predetermined time period TH, highlighting processing for generating a marker image G2 for highlighting a position of the lesion candidate region Ln and adding the generated marker image G2 to the observation image G1 based on the lesion candidate information ILn to be outputted from the lesion candidate detection unit 34b.

Note that the marker image G2 to be added by the highlighting processing of the highlighting processing unit 36a may have any form as long as the position of the lesion candidate region Ln can be presented as visual information. In other words, the highlighting processing unit 36a may perform highlighting processing using only the position information included in the lesion candidate information ILn or may perform highlighting processing using both the position information and the size information included in the lesion candidate information ILn as long as the marker image G2 for highlighting the position of the lesion candidate region Ln is generated.

The recording unit 36b is configured to record each of the observation images G1 to be sequentially outputted from the video processor 31 as a record image R1 during a measurement period of the consecutive detection time period TL. In other words, the recording unit 36b is configured to record the plurality of observation images G1 to be sequentially outputted from the video processor 31 sequentially (in chronological sequence), respectively, as a plurality of record images R1 in a period elapsed since the lesion candidate detection unit 34b in the region-of-interest detection unit 34 started to detect the lesion candidate region Ls until the detection of the lesion candidate region Ls ceases. The recording unit 36b is configured to be able to record the lesion candidate information ILn to be outputted from the lesion candidate detection unit 34b during the measurement period of the consecutive detection time period TL and the record image R1 in association with each other.

The detection start determination unit 36c as a calculation unit selects, when the region-of-interest detection unit 34 has detected the plurality of lesion candidate regions Ln, the one lesion candidate region Ls from among the regions. A method for selecting the lesion candidate region Ls from the plurality of lesion candidate regions Ln is not limited to a specific method, but an optimum method is used depending on a situation, a user preference, or the like. For example, the lesion candidate region Ln that first appears in the observation image G1 may be selected as a lesion candidate region Ls. For example, the lesion candidate region Ln highest in likelihood of a polyp may be selected as a lesion candidate region Ls from among the plurality of lesion candidate regions Ln existing within the observation image G1 at a certain time point.

The display apparatus 41 includes a monitor and the like, and is configured to be able to display a display image to be outputted from the image processing apparatus 32.

Then, a function of the present embodiment will be described. Note that a case where the lesion candidate detection unit 34b has detected two lesion candidate regions L1 and L2 and the detection start determination unit 36c has selected the lesion candidate region L1 as the lesion candidate region Ls will be described below as an example for simplicity.

(a-1) A case where lesion candidate regions L1 and L2 are simultaneously detected and the detection of lesion candidate regions L1 and L2 simultaneously ceases and a case where a consecutive detection time period TL is less than a predetermined time period TH:

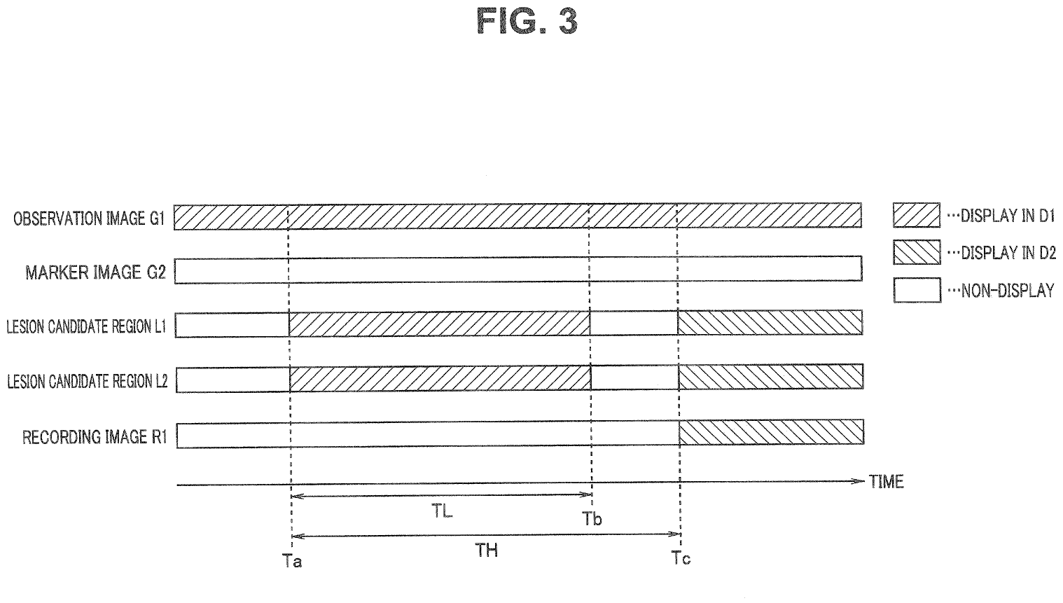

A case where the display control unit 36 performs processing corresponding to a timing chart illustrated in FIG. 3 will be described as an example. FIG. 3 is a timing chart for describing an example of processing to be performed in the image processing apparatus 32 according to the embodiment.

The endoscope 21 emits illumination light to an object, receives reflected light from the object, picks up an image of the received reflected light to generate an image pickup signal, and outputs the generated image pickup signal to the video processor 31 when respective powers to the light source driving apparatus 11 and the video processor 31 are turned on, for example.

The video processor 31 subjects the image pickup signal to be outputted from the endoscope 21 to predetermined processing to generate an observation images G1 of the object and sequentially outputs the generated observation image G1 to the image processing apparatus 32 frame by frame.

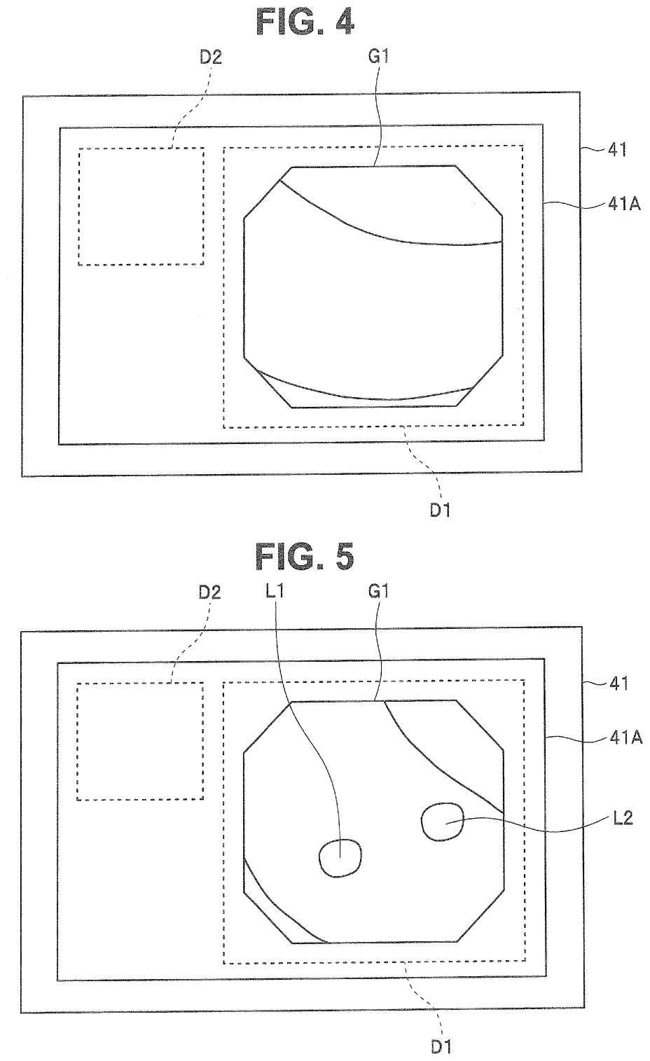

The display control unit 36 performs processing for displaying a display image in which the observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 3, for example. Note that the display region D1 is previously set as a region having a larger size than a size of a display region D2, described below, on the display screen 41A, for example. FIG. 4 is a diagram illustrating an example of a display image to be displayed on the display apparatus 41 through the processing of the image processing apparatus 32 according to the embodiment.

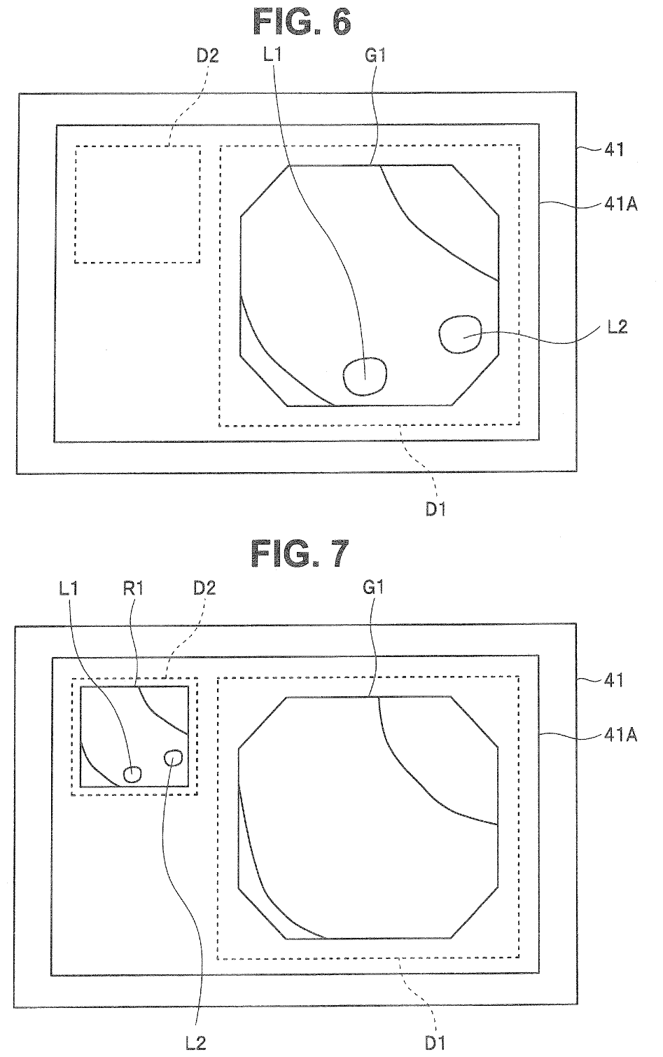

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 5 is displayed on the display screen 41A in the display apparatus 41 at a timing of the time Ta illustrated in FIG. 3, for example. According to the operation of the display control unit 36 as described above, a display image as illustrated in FIG. 6 is displayed on the display screen 41A in the display apparatus 41 at a timing immediately before a time Tb later than the time Ta illustrated in FIG. 3 is reached, for example. FIGS. 5 and 6 are diagrams respectively illustrating examples of the display images to be displayed on the display apparatus 41 through the processing of the image processing apparatus 32 according to the embodiment.

The recording unit 36b in the display control unit 36 starts processing for recording the observation image G1 to be sequentially outputted from the video processor 31 as a record image R1 while starting processing for recording the record image R1 and the lesion candidate information IL1 and IL2 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 and IL2 in association with each other is started at the timing of the time Ta illustrated in FIG. 3, for example.

The recording unit 36b in the display control unit 36 stops processing for recording each of the observation images G1 to be sequentially outputted from the video processor 31 as the record image R1 while stopping processing for recording the record image R1 and the lesion candidate information IL1 and IL2 to be outputted from the lesion candidate detection unit 34b in association with each other at a timing at which the consecutive detection time period TL has stopped being measured, that is, a timing at which the lesion candidate detection unit 34b has ceased detecting the lesion candidate region L1. According to such an operation of the recording unit 36b, processing for recording the record image R1 and the lesion candidate information IL1 and IL2 in association with each other is stopped at a timing of the time Tb illustrated in FIG. 3, for example According to the operation of the recording unit 36b as described above, the observation images G1 respectively corresponding to N (N 1) frames including at least the observation images G1 as respectively illustrated in FIGS. 5 and 6 are sequentially recorded as the record images R1 in the consecutive detection time period TL corresponding to a period from the time Ta to the time Tb illustrated in FIG. 3, for example.

The display control unit 36 starts, when the detection of the lesion candidate region L1 has ceased before the consecutive detection time period TL reaches the predetermined time period TH based on a determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the predetermined time period TH has elapsed since the lesion candidate detection unit 34b started to detect the lesion candidate region L1. According to such an operation of the display control unit 36, a display image in which the record image R1 corresponding to the observation image G1 illustrated in FIG. 6 is arranged in the display region D2, as illustrated in FIG. 7, is displayed on the display screen 41A in the display apparatus 41 at a timing of a time Tc later than the time Tb illustrated in FIG. 3, which corresponds to a timing at which the predetermined time period TH has elapsed since the lesion candidate detection unit 34b started to detect the lesion candidate region L1, for example. Note that the display region D2 is previously set as a region having a smaller size than the size of the above-described display region D1 on the display screen 41A, for example. FIG. 7 is a diagram illustrating an example of the display image to be displayed on the display apparatus 41 through the processing of the image processing apparatus 32 according to the embodiment.

The display control unit 36 performs processing for sequentially displaying the record images R1 in an order opposite to an order in which the recording unit 36b has recorded the record images R1 at and after a timing at which the predetermined time period TH has elapsed since the lesion candidate detection unit 34b started to detect the lesion candidate region L1. According to such an operation of the display control unit 36, the record images R1 respectively corresponding to the N frames sequentially recorded by the recording unit 36b, i.e., the N-th frame, the (N−1)-th frame, . . . , the second frame, and the first frame are displayed in this order in the display region D2, for example.

In other words, according to the operation of the display control unit 36 as described above, if the consecutive detection time period TL is less than the predetermined time period TH, processing for displaying the plurality of record images R1 recorded during the measurement period of the consecutive detection time period TL in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Tc illustrated in FIG. 3.

If the consecutive detection time period TL is shorter than the predetermined time period TH, as illustrated in FIG. 3, for example, there may occur a situation where the lesion candidate regions L1 and L2 move outward from inside the observation image G1 with a marker image G2 not displayed regardless of the lesion candidate regions L1 and L2 being detected by the lesion candidate detection unit 34b. Accordingly, the lesion candidate regions L1 and L2 can conceivably be easily overlooked due to user's viewing when the consecutive detection time period TL is shorter than the predetermined time period TH.

On the other hand, according to the operation of the display control unit 36 as described above, when the predetermined time period TH has elapsed since the lesion candidate region L1 started to be detected, as illustrated in FIGS. 3 and 7, for example, the record image R1 including the lesion candidate regions L1 and L2, which have already been detected by the lesion candidate detection unit 34b, can be displayed in the display region D2 on the display screen 41A. Therefore, according to the operation of the display control unit 36 as described above, overlooking of a lesion portion, which can occur in endoscope observation, can be reduced. According to the operation of the display control unit 36 as described above, the record images R1 are displayed in the display region D2 in the order opposite to the recording order by the recording unit 36b. Therefore, respective positions of the lesion candidate regions L1 and L2, which have moved outward from inside the observation image G1, can be notified to a user without the marker image G2 being added to each of the lesion candidate regions L1 and L2, for example.

Note that according to the present embodiment, the record image R1 may also continue to be recorded in a period from the time Tb to the time Tc illustrated in FIG. 3, for example, as long as the record image R1 is recorded during the measurement period of the consecutive detection time period TL.

(a-2) A case where lesion candidate regions L1 and L2 are simultaneously detected and the detection of lesion candidate regions L1 and L2 simultaneously ceases and a case where a consecutive detection time period TL is a predetermined time period TH or more:

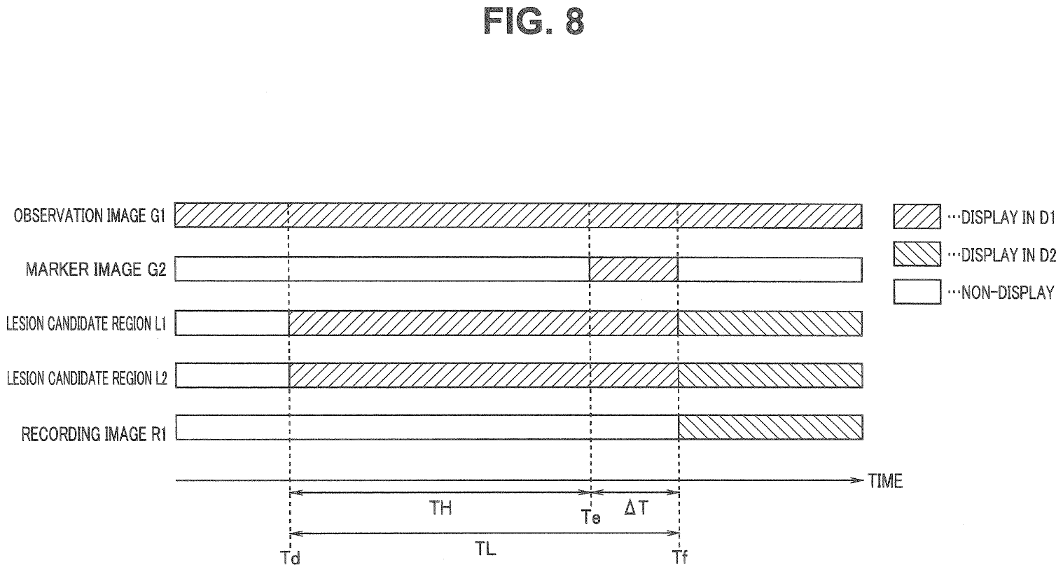

A case where the display control unit 36 performs processing corresponding to a timing chart illustrated in FIG. 8 will be described as an example. FIG. 8 is a timing chart for describing an example different from the example illustrated in FIG. 3 of processing to be performed in the image processing apparatus 32 according to the embodiment. Note that in the following, specific description relating to a portion to which processing already described, for example, can be applied is appropriately omitted for simplicity.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Td illustrated in FIG. 8, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 5 is displayed on the display screen 41A in the display apparatus 41 at a timing of the time Td illustrated in FIG. 8, for example.

The recording unit 36b in the display control unit 36 starts processing for recording each of the observation images G1 to be sequentially outputted from the video processor 31 as a record image R1 while starting processing for recording the record image R1 and the lesion candidate information IL1 and IL2 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 and IL2 in association with each other is started at the timing of the time Td illustrated in FIG. 8, for example.

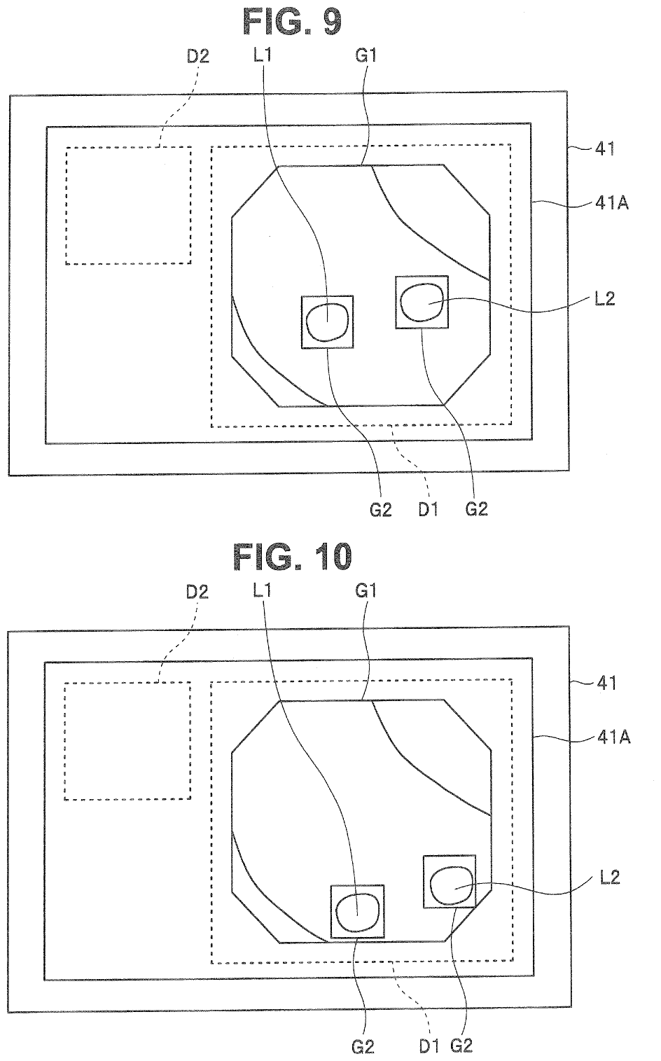

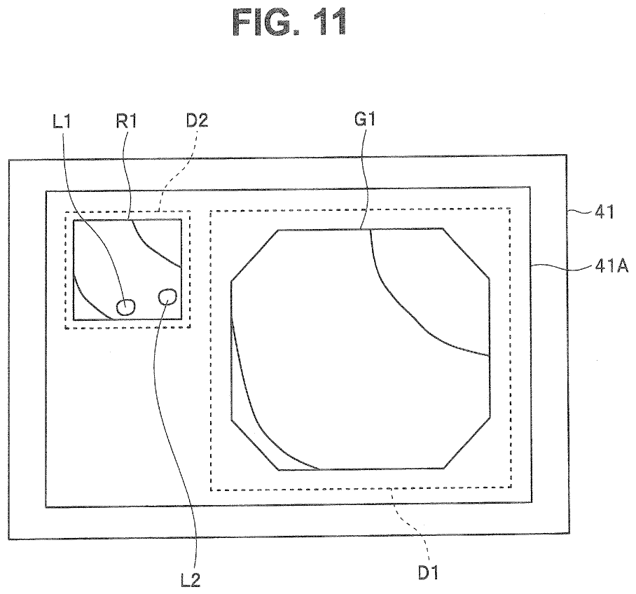

The highlighting processing unit 36a in the display control unit 36 starts highlighting processing for adding a marker image G2 for highlighting respective positions of the lesion candidate regions L1 and L2 detected by the lesion candidate detection unit 34b to the observation image G1 at a timing at which the predetermined time period TH has elapsed since the consecutive detection time period TL started to be measured based on a determination result to be outputted from the consecutive detection determination unit 35. According to such an operation of the highlighting processing unit 36a, a display image as illustrated in FIG. 9 is displayed on the display screen 41A in the display apparatus 41 at a timing of a time Te illustrated in FIG. 8, which corresponds to a timing at which the predetermined time period TH has elapsed since the lesion candidate detection unit 34b started to detect the lesion candidate region L1, for example. According to the operation of the highlighting processing unit 36a as described above, a display image as illustrated in FIG. 10 is displayed on the display screen 41A in the display apparatus 41 at a timing immediately before a time Tf later than the time Te illustrated in FIG. 8 is reached. Note that a case where respective rectangular frames surrounding peripheries of the lesion candidate regions L1 and L2 are each added as the marker image G2, as illustrated in FIGS. 9 and 10, by the highlighting processing of the highlighting processing unit 36a will be described below as an example. FIGS. 9 and 10 are diagrams respectively illustrating examples of the display images to be displayed on the display apparatus 41 through the processing of the image processing apparatus 32 according to the embodiment.

The recording unit 36b in the display control unit 36 stops processing for recording each of the observation images G1 to be sequentially outputted from the video processor 31 as the record image R1 while stopping processing for recording the record image R1 and the lesion candidate information IL to be outputted from the lesion candidate detection unit 34b in association with each other at a timing at which the consecutive detection time period TL has stopped being measured, that is, a timing at which the lesion candidate detection unit 34b has ceased detecting the lesion candidate region L1. According to such an operation of the recording unit 36b, processing for recording the record image R1 and the lesion candidate information IL in association with each other is stopped at a timing of the time Tf illustrated in FIG. 8, for example. According to the operation of the recording unit 36b as described above, the observation images G1 respectively corresponding to P (P 1) frames including at least the observation images G1 as respectively illustrated in FIGS. 9 and 10 are sequentially recorded as the record images R1 in the consecutive detection time period TL corresponding to a period from the time Td to the time Tf illustrated in FIG. 8, for example.

The display control unit 36 starts, when the detection of the lesion candidate region L1 has ceased after the consecutive detection time period TL has reached the predetermined time period TH or more based on the determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the detection of the lesion candidate region L1 has ceased. According to such an operation of the display control unit 36, a display image in which the record image R1 corresponding to the observation image G1 illustrated in FIG. 10, as illustrated in FIG. 11, is arranged in the display region D2 is displayed on the display screen 41A in the display apparatus 41 at the timing of the time Tf illustrated in FIG. 8, for example. FIG. 11 is a diagram illustrating an example of a display image to be displayed on the display apparatus 41 through the processing of the image processing apparatus 32 according to the embodiment.

The display control unit 36 performs processing for sequentially displaying the record images R1 in an order opposite to an order in which the recording unit 36b has recorded the record images R1 at and after a timing at which the lesion candidate detection unit 34b has ceased detecting the lesion candidate region L1. According to such an operation of the display control unit 36, the record images R1 respectively corresponding to the P frames sequentially recorded by the recording unit 36b, i.e., the P-th frame, the (P−1)-th frame, . . . , the second frame, and the first frame are displayed in this order in the display region D2, for example.

In other words, according to the operation of the display control unit 36 as described above, if the consecutive detection time period TL is the predetermined time period TH or more, processing for displaying the plurality of record images R1 recorded during the measurement period of the consecutive detection time period TL in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Tf illustrated in FIG. 8.

If a difference time period ΔT between the consecutive detection time period TL and the predetermined time period TH, which corresponds to the period from the time Te to the time Tf illustrated in FIG. 8, for example, is significantly short, there may occur a situation where the lesion candidate region L1 highlighted by the marker image G2 moves outward from inside the observation image G1 with the marker image G2 momentarily displayed in the display region D1 being visually unrecognizable by a user. Accordingly, the lesion candidate regions L1 and L2 can conceivably be easily overlooked due to user's viewing even when the difference time period ΔT is significantly short.

On the other hand, according to the operation of the display control unit 36 as described above, when the detection of the lesion candidate region L1 has ceased after the predetermined time period TH has elapsed, as illustrated in FIGS. 8 and 11, for example, the record image R1 including the lesion candidate regions L1 and L2, which have already been detected by the lesion candidate detection unit 34b, can be displayed in the display region D2 on the display screen 41A. Therefore, according to the operation of the display control unit 36 as described above, overlooking of a lesion portion, which can occur in endoscope observation, can be reduced. According to the operation of the display control unit 36 as described above, the record images R1 are displayed in the display region D2 in the order opposite to the recording order by the recording unit 36b. Therefore, respective positions of the lesion candidate regions L1 and L2, which have moved outward from inside the observation image G1, can be notified to the user after the marker image G2 has been added to each of the lesion candidate regions L1 and L2 only momentarily, for example.

(b-1) A case where lesion candidate regions L1 and L2 are simultaneously detected and the detection of the lesion candidate region L1 ceases before the detection of the lesion candidate region L2 and a case where a consecutive detection time period TL is less than a predetermined time period TH:

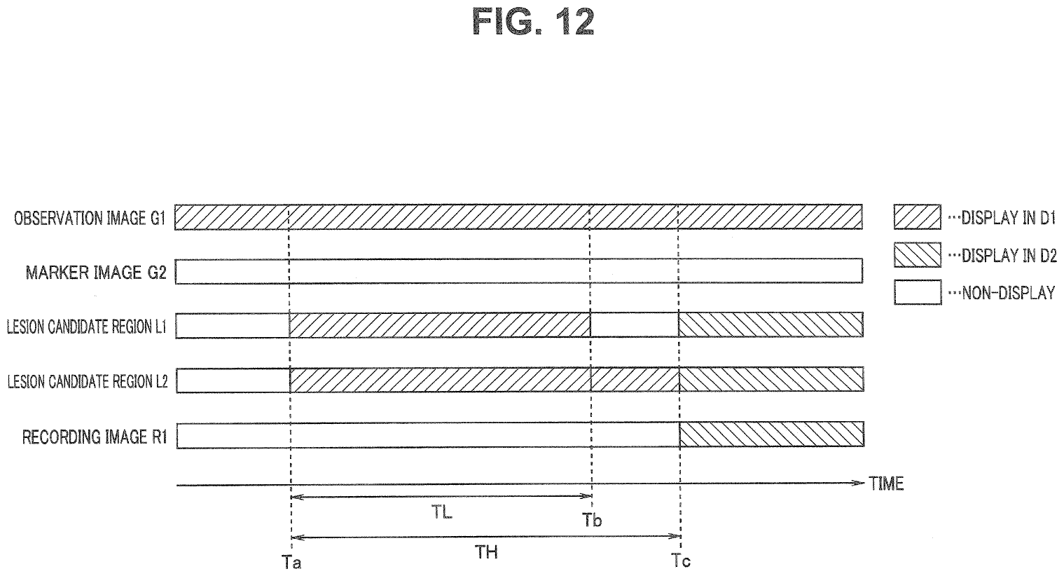

A case where the display control unit 36 performs processing corresponding to a timing chart illustrated in FIG. 12 will be described as an example. FIG. 12 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 12, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 5 is displayed on the display screen 41A in the display apparatus 41 at a timing of the time Ta illustrated in FIG. 12, for example.

The recording unit 36b in the display control unit 36 starts processing for recording each of the observation images G1 to be sequentially outputted from the video processor 31 as a record image R1 while starting processing for recording the record image R1 and the lesion candidate information IL1 and IL2 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 and IL2 in association with each other is started at the timing of the time Ta illustrated in FIG. 12, for example.

The recording unit 36b in the display control unit 36 stops processing for recording each of the observation images G1 to be sequentially outputted from the video processor 31 as the record image R1 while stopping processing for recording the record image R1 and the lesion candidate information IL1 and IL2 to be outputted from the lesion candidate detection unit 34b in association with each other at a timing at which the consecutive detection time period TL has stopped being measured, that is, a timing at which the lesion candidate detection unit 34b has ceased detecting the lesion candidate region L1. According to such an operation of the recording unit 36b, processing for recording the record image R1 and the lesion candidate information IL1 and IL2 in association with each other is stopped at a timing of a time Tb illustrated in FIG. 12, for example. According to the operation of the recording unit 36b as described above, the observation images G1 respectively corresponding to Q (Q 1) frames including at least the observation images G1 as respectively illustrated in FIGS. 5 and 6 are sequentially recorded as the record images R1 in the consecutive detection time period TL corresponding to a period from the time Ta to the time Tb illustrated in FIG. 12, for example.

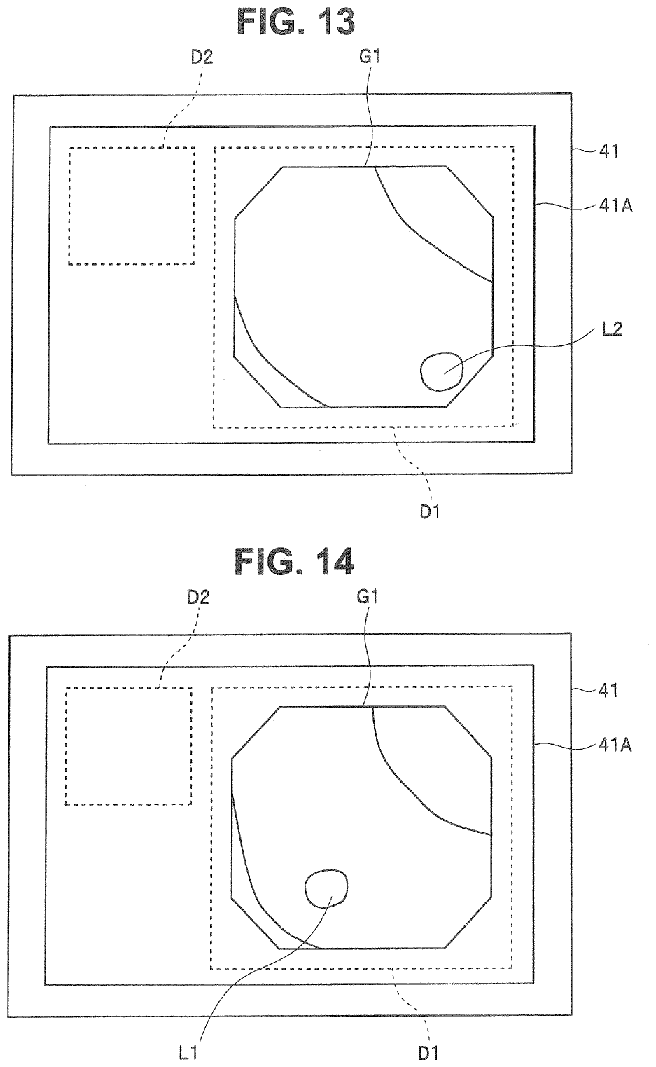

The display control unit 36 also performs processing for displaying a display image in which the observation image G1 including the lesion candidate region L2 is arranged in the display region D1 on the display screen 41A until the predetermined time period TH has elapsed since the lesion candidate detection unit 34b started to detect the lesion candidate region L1 from a timing at which the consecutive detection time period TL has stopped being measured, that is, a timing at which the lesion candidate detection unit 34b has ceased detecting the lesion candidate region L1. According to such an operation of the display control unit 36, a display image as illustrated in FIG. 13 is displayed on the display screen 41A in the display apparatus 41 at any timing between the time Tb to a time Tc illustrated in FIG. 12, for example.

The display control unit 36 starts, when the detection of the lesion candidate region L1 has ceased before the consecutive detection time period TL reaches the predetermined time period TH based on a determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the predetermined time period TH has elapsed since the lesion candidate detection unit 34b started to detect the lesion candidate region L1. According to such an operation of the display control unit 36, a display image in which the record image R1 corresponding to the observation image G1 illustrated in FIG. 6 is arranged in the display region D2, as illustrated in FIG. 7, is displayed on the display screen 41A in the display apparatus 41 at a timing of the time Tc later than the time Tb illustrated in FIG. 12, which corresponds to a timing at which the predetermined time period TH has elapsed since the lesion candidate detection unit 34b started to detect the lesion candidate region L1, for example.

The display control unit 36 performs processing for sequentially displaying the record images R1 in an order opposite to an order in which the recording unit 36b has recorded the record images R1 at and after a timing at which the predetermined time period TH has elapsed since the lesion candidate detection unit 34b started to detect the lesion candidate region L1. According to such an operation of the display control unit 36, the record images R1 respectively corresponding to the Q frames sequentially recorded by the recording unit 36b, i.e., the Q-th frame, the (Q−1)-th frame, . . . , the second frame, and the first frame are displayed in this order in the display region D2, for example.

In other words, according to the operation of the display control unit 36 as described above, if the consecutive detection time period TL is less than the predetermined time period TH, processing for displaying the plurality of record images R1 recorded during the measurement period of the consecutive detection time period TL in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Tc illustrated in FIG. 12.

If the consecutive detection time period TL is shorter than the predetermined time period TH, as illustrated in FIG. 3, for example, there may occur a situation where the lesion candidate regions L1 and L2 move outward from inside the observation image G1 with a marker image G2 not displayed regardless of the lesion candidate regions L1 and L2 being detected by the lesion candidate detection unit 34b. Accordingly, the lesion candidate regions L1 and L2 can conceivably be easily overlooked due to user's viewing when the consecutive detection time period TL is shorter than the predetermined time period TH.

On the other hand, according to the operation of the display control unit 36 as described above, when the predetermined time period TH has elapsed since the lesion candidate region L1 started to be detected, as illustrated in FIGS. 3 and 7, for example, the record image R1 including the lesion candidate regions L1 and L2, which have already been detected by the lesion candidate detection unit 34b, can be displayed in the display region D2 on the display screen 41A. Therefore, according to the operation of the display control unit 36 as described above, overlooking of a lesion portion, which can occur in endoscope observation, can be reduced. According to the operation of the display control unit 36 as described above, the record images R1 are displayed in the display region D2 in the order opposite to the recording order by the recording unit 36b. Therefore, respective positions of the lesion candidate regions L1 and L2, which have moved outward from inside the observation image G1, can be notified to a user without the marker image G2 being added to each of the lesion candidate regions L1 and L2, for example.

Further, the lesion candidate region L2 also continues to be displayed in the display region D1 until the predetermined time period TH has elapsed since the lesion candidate region L1 started to be detected even after the detection of the lesion candidate region L1 has ceased. Therefore, the overlooking of the lesion candidate region L2 due to user's viewing can be further reduced.

Note that if the detection of the lesion candidate region L2 ceases before the detection of the lesion candidate region L1, the lesion candidate region L2 disappears from the display region D1 at a timing before the time Tb illustrated in FIG. 12. At the timing of the time Tb illustrated in FIG. 12, for example, a display image in which the observation image G1 is arranged in the display region D1, as illustrated in FIG. 14, is displayed on the display screen 41A in the display apparatus 41. FIG. 14 is a diagram illustrating an example of the display image to be displayed on the display apparatus 41 through the processing of the image processing apparatus 32 according to the embodiment. However, when the predetermined time period TH has elapsed since the lesion candidate region L1 started to be detected, the record image R1 including the lesion candidate region L2 is displayed in the display region D2 on the display screen 41A, as illustrated in FIG. 7. Therefore, the overlooking of the lesion candidate region L2 due to user's viewing can be reduced.

(b-2) A case where lesion candidate regions L1 and L2 are simultaneously detected and the detection of the lesion candidate region L1 ceases before the detection of the lesion candidate region L2 and a case where a consecutive detection time period TL is a predetermined time period TH or more:

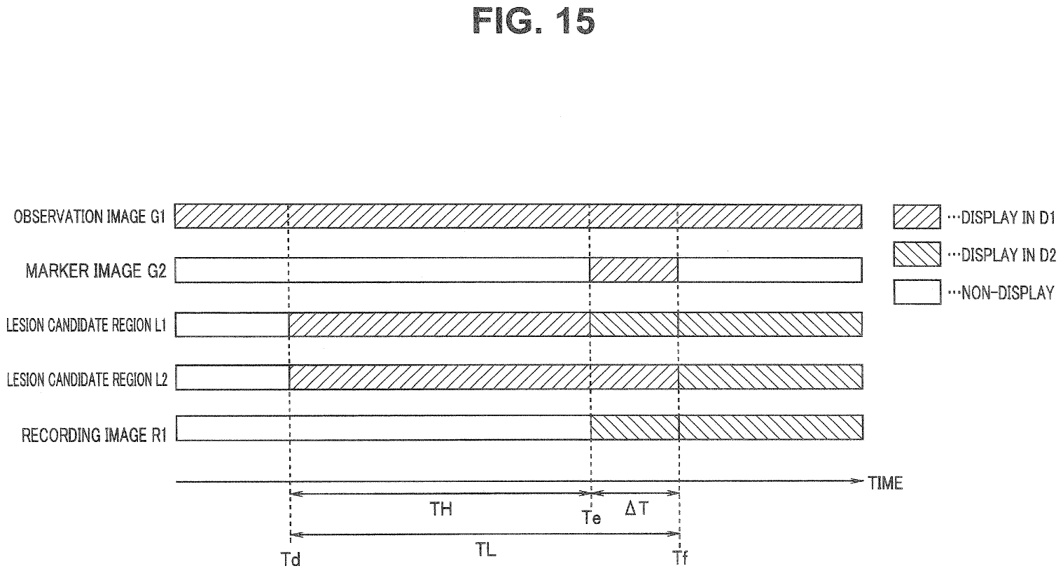

A case where the display control unit 36 performs processing corresponding to a timing chart illustrated in FIG. 15 will be described as an example. FIG. 15 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Td illustrated in FIG. 15, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 5 is displayed on the display screen 41A in the display apparatus 41 at a timing of the time Td illustrated in FIG. 15, for example

The recording unit 36b in the display control unit 36 starts processing for recording each of observation images G1 to be sequentially outputted from the video processor 31 as a record image R1 while starting processing for recording the record image R1 and the lesion candidate information IL1 and IL2 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 and IL2 in association with each other is started at the timing of the time Td illustrated in FIG. 15, for example.

The highlighting processing unit 36a in the display control unit 36 starts highlighting processing for adding a marker image G2 for highlighting respective positions of the lesion candidate regions L1 and L2 detected by the lesion candidate detection unit 34b to the observation image G1 at a timing at which the predetermined time period TH has elapsed since the consecutive detection time period TL started to be measured based on a determination result to be outputted from the consecutive detection determination unit 35. Note that at a timing of a time Te illustrated in FIG. 15, the detection of the lesion candidate region L1 has ceased. Therefore, the marker image G2 is added only to the remaining lesion candidate region L2.

The recording unit 36b in the display control unit 36 stops processing for recording each of the observation images G1 to be sequentially outputted from the video processor 31 as the record image R1 while stopping processing for recording the record image R1 and the lesion candidate information IL to be outputted from the lesion candidate detection unit 34b in association with each other at a timing at which the lesion candidate detection unit 34b has ceased detecting either one of the lesion candidate regions L1 and L2 after the predetermined time period TH has elapsed since the consecutive detection time period TL started to be measured. According to such an operation of the recording unit 36b, processing for recording the record image R1 and the lesion candidate information IL in association with each other is stopped at the timing of the time Te illustrated in FIG. 15, for example. According to the operation of the recording unit 36b as described above, the observation images G1 respectively corresponding to S (S 1) frames are sequentially recorded, respectively, as the record images R1 in a period from the time Td to the time Te illustrated in FIG. 15, for example.

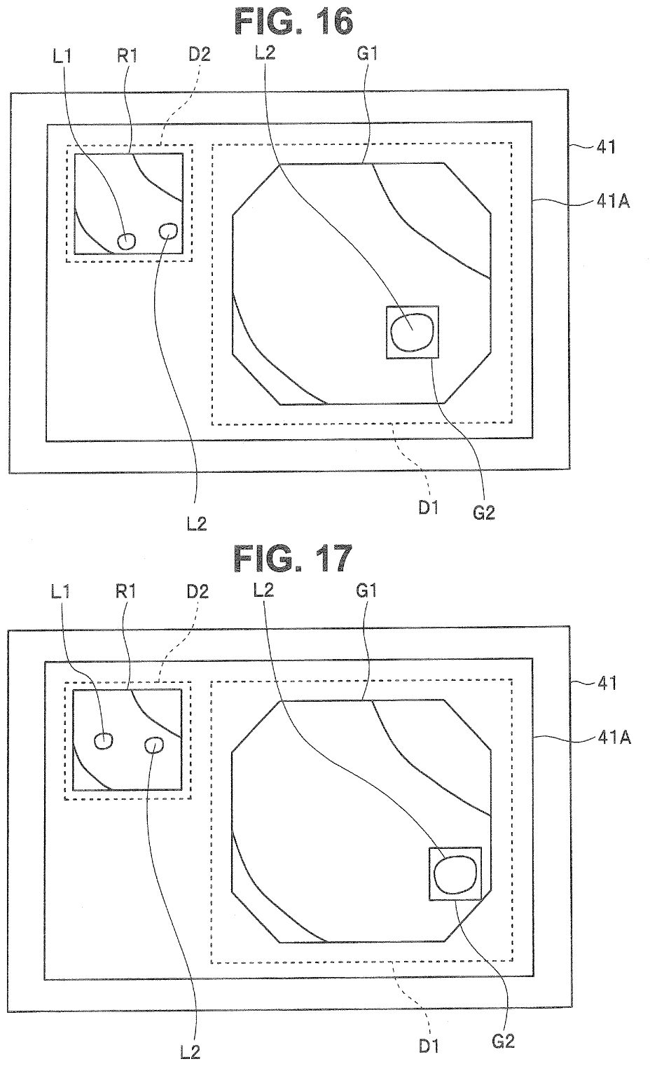

The display control unit 36 starts processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the lesion candidate detection unit 34b has ceased detecting either one of the lesion candidate regions L1 and L2, that is, at the time Te illustrated in FIG. 15 after the predetermined time period TH has elapsed since the consecutive detection time period TL started to be measured. According to such an operation of the display control unit 36, a display image in which the record image R1 is arranged in the display region D2, as illustrated in FIG. 16, is displayed on the display screen 41A in the display apparatus 41 at the timing of the time Te illustrated in FIG. 15, for example. FIG. 16 is a diagram illustrating an example of a display image to be displayed on the display apparatus 41 through the processing of the image processing apparatus 32 according to the embodiment.

The display control unit 36 performs processing for sequentially displaying the record images R1 in an order opposite to an order in which the recording unit 36b has recorded the record images R1 at and after a timing at which the lesion candidate detection unit 34b has ceased detecting the lesion candidate region L1. According to such an operation of the display control unit 36, the record images R1 respectively corresponding to the S frames sequentially recorded by the recording unit 36b, i.e., the S-th frame, the (S−1)-th frame, . . . , the second frame, and the first frame are displayed in this order in the display region D2, for example.

On the other hand, according to the operation of the highlighting processing unit 36a, a rectangular frame surrounding a periphery of the lesion candidate region L2 is added as the marker image G2 to the observation image G1 to be displayed in the display region D1 on the display screen 41A at and after the timing of the time Te illustrated in FIG. 15, which corresponds to a timing at which the predetermined time period TH has elapsed since the lesion candidate detection unit 34b started to detect the lesion candidate region L1, for example. In other words, the observation image G1 including the lesion candidate region L2 to which the marker image G2 is added is displayed in the display region D1 on the display screen 41A in a period from the time Te illustrated in FIG. 15 to a time Tf at which the detection of the lesion candidate region L2 has ceased. At a timing of the time Tf illustrated in FIG. 15, for example, a display image in which the observation image G1 including the lesion candidate region L2 to which the marker image G2 is added is arranged in the display region D1 and the record image R1 is arranged in the display region D2, as illustrated in FIG. 17, is displayed on the display screen 41A in the display apparatus 41. FIG. 17 is a diagram illustrating an example of the display image to be displayed on the display apparatus 41 through the processing of the image processing apparatus 32 according to the embodiment.

In other words, according to the operation of the display control unit 36 as described above, if the consecutive detection time period TL is the predetermined time period TH or more, processing for displaying the plurality of record images R1 recorded during the predetermined time period TH in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Te illustrated in FIG. 15.

Accordingly, even when the consecutive detection time period TL is the predetermined time period TH or more and when the detection of the lesion candidate region L1 ceases before the consecutive detection time period TL, the record image R1 including the lesion candidate regions L1 and L2, which have already been detected by the lesion candidate detection unit 34b, is displayed in the display region D2 on the display screen 41A from a timing at which the detection of the lesion candidate region L1 has ceased so that overlooking of a lesion portion, which can occur in endoscope observation, can be reduced.

Note that a timing at which processing for recording each of the observation images G1 to be sequentially outputted from the video processor 31 as the record image R1 in the recording unit 36b is stopped may be a timing at which the consecutive detection time period TL finishes being measured or a timing at which the detection of all the lesion candidate regions L1 and L2 existing within the observation image G1 ceases. A timing at which the record image R1 recorded by the recording unit 36b starts to be displayed may also be a timing at which the consecutive detection time period TL finishes being measured or a timing at which the detection of all the lesion candidate regions L1 and L2 existing within the observation image G1 ceases.

(c-1) A case where a lesion candidate region L1 is detected before a lesion candidate region L2 and the detection of the lesion candidate regions L1 and L2 simultaneously ceases and a case where a consecutive detection time period TL is less than a predetermined time period TH:

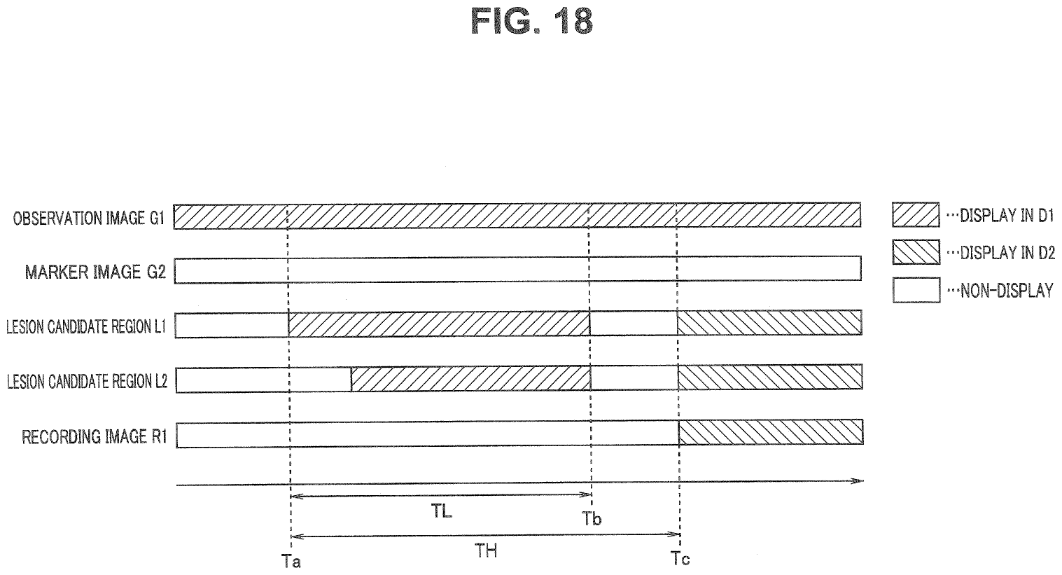

A case where the display control unit 36 performs processing corresponding to a timing chart illustrated in FIG. 18 will be described as an example. FIG. 18 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 18, for example.

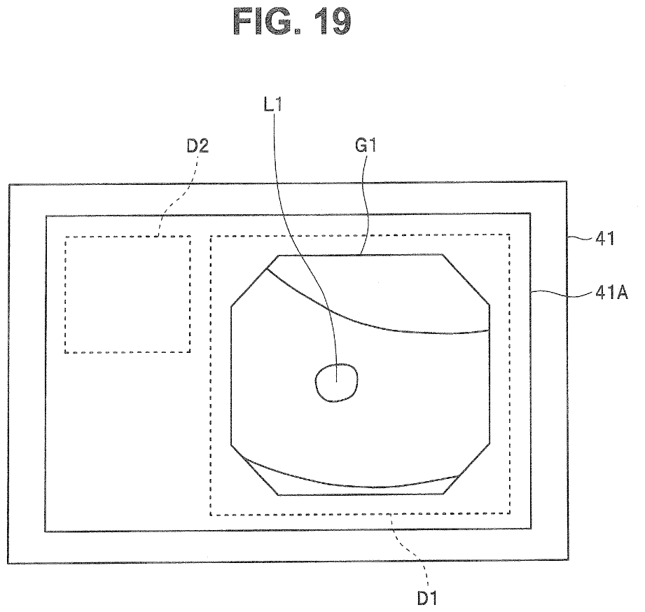

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of the time Ta illustrated in FIG. 18, for example. FIG. 19 is a diagram illustrating an example of a display image displayed on the display apparatus 41 through the processing of the image processing apparatus 32 according to the embodiment.

The recording unit 36b in the display control unit 36 starts processing for recording each of the observation images G1 to be sequentially outputted from the video processor 31 as a record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is started at the timing of the time Ta illustrated in FIG. 18, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image RE

The recording unit 36b in the display control unit 36 stops processing for recording each of the observation images G1 to be sequentially outputted from the video processor 31 as the record image R1 while stopping processing for recording the record image R1 and the lesion candidate information IL1 and IL2 to be outputted from the lesion candidate detection unit 34b in association with each other at a timing at which the consecutive detection time period TL has stopped being measured, that is, a timing at which the lesion candidate detection unit 34b has ceased detecting the lesion candidate region L1. According to such an operation of the recording unit 36b, processing for recording the record image R1 and the lesion candidate information IL1 and IL2 in association with each other is stopped at a timing of a time Tb illustrated in FIG. 18, for example. According to the operation of the recording unit 36b as described above, the observation images G1 respectively corresponding to U (U 1) frames including at least the observation images G1 as respectively illustrated in FIGS. 19 and 5 are sequentially recorded as the record images R1 in the consecutive detection time period TL corresponding to a period from the time Ta to the time Tb illustrated in FIG. 18, for example.

The display control unit 36 starts, when the detection of the lesion candidate region L1 has ceased before the consecutive detection time period TL reaches the predetermined time period TH based on a determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the predetermined time period TH has elapsed since the lesion candidate detection unit 34b started to detect the lesion candidate region L1. According to such an operation of the display control unit 36, a display image as illustrated in FIG. 7 is displayed on the display screen 41A in the display apparatus 41 at a timing of a time Tc later than the time Tb illustrated in FIG. 18, which corresponds to a timing at which the predetermined time period TH has elapsed since the lesion candidate detection unit 34b started to detect the lesion candidate region L1, for example.

The display control unit 36 performs processing for sequentially displaying the record images R1 in an order opposite to an order in which the recording unit 36b has recorded the record images R1 at and after a timing at which the predetermined time period TH has elapsed since the lesion candidate detection unit 34b started to detect the lesion candidate region L1. According to such an operation of the display control unit 36, the record images R1 respectively corresponding to the U frames sequentially recorded by the recording unit 36b, i.e., the U-th frame, the (U−1)-th frame, . . . , the second frame, and the first frame are displayed in this order in the display region D2, for example.

In other words, according to the operation of the display control unit 36 as described above, if the consecutive detection time period TL is less than the predetermined time period TH, processing for displaying the plurality of record images R1 recorded during the measurement period of the consecutive detection time period TL in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Tc illustrated in FIG. 18.

Accordingly, if the consecutive detection time period TL is shorter than the predetermined time period TH and further if a detection time period of the lesion candidate region L2 is shorter than the consecutive detection time period TL based on the lesion candidate region L1, there may occur a situation where the lesion candidate regions L1 and L2 move outward from inside the observation image G1 with a marker image G2 not displayed in the observation image G1. Accordingly, the lesion candidate region L2 can conceivably be more easily overlooked due to user's viewing.

On the other hand, according to the operation of the display control unit 36 as described above, when the predetermined time period TH has elapsed since the lesion candidate region L1 started to be detected, as illustrated in FIGS. 18 and 7, for example, the record image R1 including the lesion candidate regions L1 and L2, which have already been detected by the lesion candidate detection unit 34b, can be displayed in the display region D2 on the display screen 41A. Therefore, according to the operation of the display control unit 36 as described above, overlooking of a lesion portion, which can occur in endoscope observation, can be reduced. According to the operation of the display control unit 36 as described above, the record images R1 are displayed in the display region D2 in the order opposite to the recording order by the recording unit 36b. Therefore, respective positions of the lesion candidate regions L1 and L2, which have moved outward from inside the observation image G1, can be notified to a user without the marker image G2 being added to each of the lesion candidate regions L1 and L2, for example.

(c-2) A case where a lesion candidate region L1 is detected before a lesion candidate region L2 and the detection of the lesion candidate regions L1 and L2 simultaneously ceases and a case where a consecutive detection time period TL is a predetermined time period TH or more:

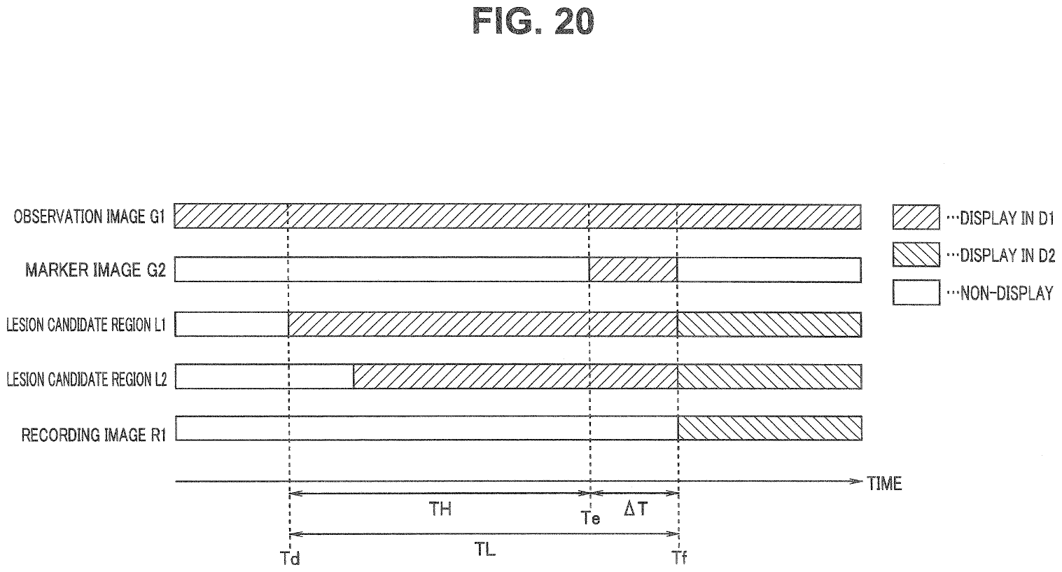

A case where the display control unit 36 performs processing corresponding to a timing chart illustrated in FIG. 20 will be described as an example. FIG. 20 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 18, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of a time Td illustrated in FIG. 20, for example.

The recording unit 36b in the display control unit 36 starts processing for recording each of the observation images G1 to be sequentially outputted from the video processor 31 as a record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is started at the timing of the time Td illustrated in FIG. 20, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image RE

The highlighting processing unit 36a in the display control unit 36 starts highlighting processing for adding a marker image G2 for highlighting respective positions of the lesion candidate regions L1 and L2 detected by the lesion candidate detection unit 34b to the observation image G1 at a timing at which the predetermined time period TH has elapsed since the consecutive detection time period TL started to be measured based on a determination result to be outputted from the consecutive detection determination unit 35. According to such an operation of the highlighting processing unit 36a, a display image as illustrated in FIG. 9 is displayed on the display screen 41A in the display apparatus 41 at a timing of a time Te illustrated in FIG. 20, which corresponds to a timing at which the predetermined time period TH has elapsed since the lesion candidate detection unit 34b started to detect the lesion candidate region L1, for example. According to the operation of the highlighting processing unit 36a as described above, a display image as illustrated in FIG. 10 is displayed on the display screen 41A in the display apparatus 41 at a timing immediately before a time Tf later than the time Te illustrated in FIG. 20 is reached.

The recording unit 36b in the display control unit 36 stops processing for recording each of the observation images G1 to be sequentially outputted from the video processor 31 as the record image R1 while stopping processing for recording the record image R1 and the lesion candidate information IL1 to be outputted from the lesion candidate detection unit 34b in association with each other at a timing at which the consecutive detection time period TL has stopped being measured, that is, a timing at which the lesion candidate detection unit 34b has ceased detecting the lesion candidate region L1. The observation images G1 respectively corresponding to V (V 1) frames including at least the observation images G1 as respectively illustrated in FIGS. 9 and 10 are sequentially recorded as the record images R1 in the consecutive detection time period TL corresponding to a period from the time Td to the time Tf illustrated in FIG. 20, for example.

The display control unit 36 starts, when the detection of the lesion candidate region L1 has ceased after the consecutive detection time period TL has reached the predetermined time period TH or more based on the determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the detection of the lesion candidate region L1 has ceased.

According to such an operation of the display control unit 36, a display image in which the record image R1 corresponding to the observation image G1 illustrated in FIG. 10 is arranged in the display region D2, as illustrated in FIG. 11, is displayed on the display screen 41A in the display apparatus 41 at a timing of the time Tf illustrated in FIG. 20, for example.

The display control unit 36 performs processing for sequentially displaying the record images R1 in an order opposite to an order in which the recording unit 36b has recorded the record images R1 at and after a timing at which the lesion candidate detection unit 34b has ceased detecting the lesion candidate region L1. According to such an operation of the display control unit 36, the record images R1 respectively corresponding to the V frames sequentially recorded by the recording unit 36b, i.e., the V-th frame, the (V−1)-th frame, . . . , the second frame, and the first frame are displayed in this order in the display region D2, for example.

In other words, according to the operation of the display control unit 36 as described above, if the consecutive detection time period TL is the predetermined time period TH or more, processing for displaying the plurality of record images R1 recorded during the measurement period of the consecutive detection time period TL in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Tf illustrated in FIG. 20.

If a difference time period ΔT between the consecutive detection time period TL and the predetermined time period TH, which corresponds to a period from the time Te to the time Tf illustrated in FIG. 8, for example, is significantly short, there may occur a situation where the lesion candidate region L1 highlighted by the marker image G2 moves outward from inside the observation image G1 with the marker image G2 momentarily displayed in the display region D1 being visually unrecognizable by a user. Accordingly, the lesion candidate regions L1 and L2 can conceivably be easily overlooked due to user's viewing even when the difference time period ΔT is significantly short. Further, if a detection time period of the lesion candidate region L2 is shorter than the consecutive detection time period TL based on the lesion candidate region L1, the lesion candidate region L2 may be more easily overlooked.

On the other hand, when the detection of the lesion candidate region L1 has ceased after the predetermined time period TH has elapsed, the record image R1 including the lesion candidate regions L1 and L2, which have already been detected by the lesion candidate detection unit 34b, can be displayed in the display region D2 on the display screen 41A. Therefore, according to the operation of the display control unit 36 as described above, overlooking of a lesion portion, which can occur in endoscope observation, can be reduced. According to the operation of the display control unit 36 as described above, the record images R1 are displayed in the display region D2 in the order opposite to the recording order by the recording unit 36b. Therefore, respective positions of the lesion candidate regions L1 and L2, which have moved outward from inside the observation image G1, can be notified to the user after the marker image G2 has been only momentarily added to each of the lesion candidate regions L1 and L2, for example.

(d-1) A case where a lesion candidate region L1 is detected before a lesion candidate region L2 and the detection of the lesion candidate region L1 ceases before the detection of the lesion candidate region L2 and a case where a consecutive detection time period TL is less than a predetermined time period TH:

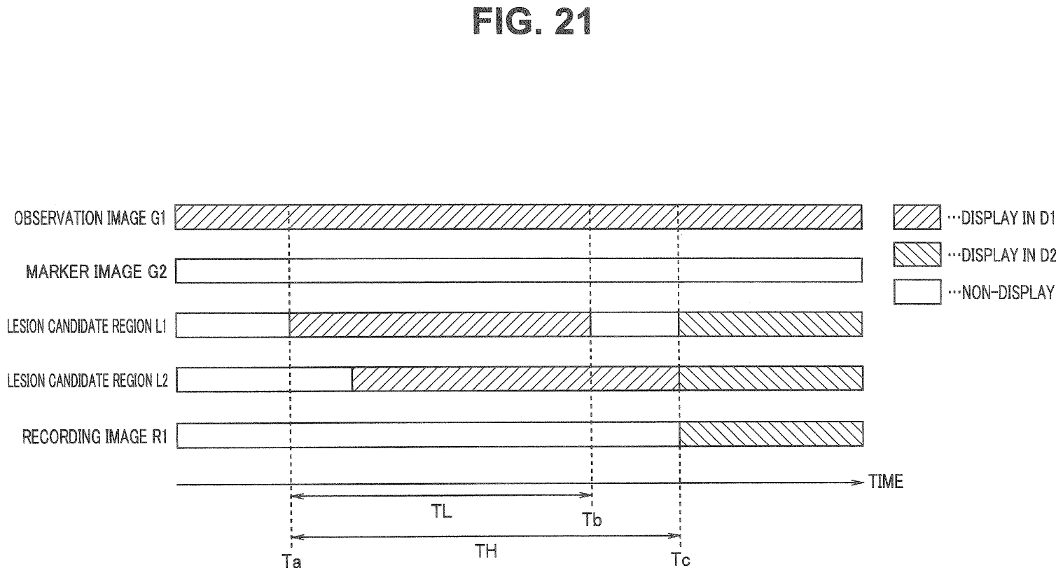

A case where the display control unit 36 performs processing corresponding to a timing chart illustrated in FIG. 21 will be described as an example. FIG. 21 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 21, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of the time Ta illustrated in FIG. 21, for example.

The recording unit 36b in the display control unit 36 starts processing for recording each of the observation images G1 to be sequentially outputted from the video processor 31 as a record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is stated at the timing of the time Ta illustrated in FIG. 21, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image RE

The recording unit 36b in the display control unit 36 stops processing for recording each of the observation images G1 to be sequentially outputted from the video processor 31 as the record image R1 while stopping processing for recording the record image R1 and the lesion candidate information IL1 and IL2 to be outputted from the lesion candidate detection unit 34b in association with each other at a timing at which the consecutive detection time period TL has stopped being measured. For example, processing for recording the record image R1 and the lesion candidate information IL1 and IL2 in association with each other is stopped at a timing of a time Tb illustrated in FIG. 21, for example. According to the operation of the recording unit 36b as described above, the observation images G1 respectively corresponding to W (W≥1) frames are sequentially recorded, respectively, as the record images R1 in the consecutive detection time period TL corresponding to a period from the time Ta to the time Tb illustrated in FIG. 21, for example.

The display control unit 36 also performs processing for displaying a display image in which the observation image G1 including the lesion candidate region L2 is arranged in the display region D1 on the display screen 41A until the predetermined time period TH has elapsed since the lesion candidate detection unit 34b started to detect the lesion candidate region L1 from a timing at which the consecutive detection time period TL has stopped being measured, that is, a timing at which the lesion candidate detection unit 34b has ceased detecting the lesion candidate region L1. According to such an operation of the display control unit 36, a display image as illustrated in FIG. 13 is displayed on the display screen 41A in the display apparatus 41 at any timing between the time Tb to a time Tc illustrated in FIG. 21, for example.

The display control unit 36 starts, when the detection of the lesion candidate region L1 has ceased before the consecutive detection time period TL reaches the predetermined time period TH based on a determination result to be outputted from the consecutive detection determination unit 35, processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the predetermined time period TH has elapsed since the lesion candidate detection unit 34b started to detect the lesion candidate region L1. According to such an operation of the display control unit 36, a display image as illustrated in FIG. 7 is displayed on the display screen 41A in the display apparatus 41 at a timing of the time Tc later than the time Tb illustrated in FIG. 21, which corresponds to a timing at which the predetermined time period TH has elapsed since the lesion candidate detection unit 34b started to detect the lesion candidate region L1, for example.

The display control unit 36 performs processing for sequentially displaying the record images R1 in an order opposite to an order in which the recording unit 36b has recorded the record images R1 at and after a timing at which the predetermined time period TH has elapsed since the lesion candidate detection unit 34b started to detect the lesion candidate region L1. According to such an operation of the display control unit 36, the record images R1 respectively corresponding to the W frames sequentially recorded by the recording unit 36b, i.e., the W-th frame, the (W−1)-th frame, . . . , the second frame, and the first frame are displayed in this order in the display region D2, for example.

Accordingly, there may occur a situation where the lesion candidate regions L1 and L2 move outward from inside the observation image G1 with a marker image G2 not displayed in the observation image G1. Accordingly, the lesion candidate region L2 can conceivably be more easily overlooked due to user's viewing.

On the other hand, the record images R1 are displayed in the display region D2 in the order opposite to the recording order by the recording unit 36b. Therefore, respective positions of the lesion candidate regions L1 and L2, which have moved outward from inside the observation image G1, can be notified to a user without the marker image G2 being added to each of the lesion candidate regions L1 and L2, for example.

Further, the lesion candidate region L2 continues to be displayed in the display region D1 until the predetermined time period TH has elapsed since the lesion candidate region L1 started to be detected even after the detection of the lesion candidate region L1 has ceased. Therefore, the overlooking of the lesion candidate region L2 due to user's viewing can be further reduced.

(d-2) A case where a lesion candidate region L1 is detected before a lesion candidate region L2 and the detection of the lesion candidate region L1 ceases before the detection of the lesion candidate region L2 and a case where a consecutive detection time period TL is a predetermined time period TH or more:

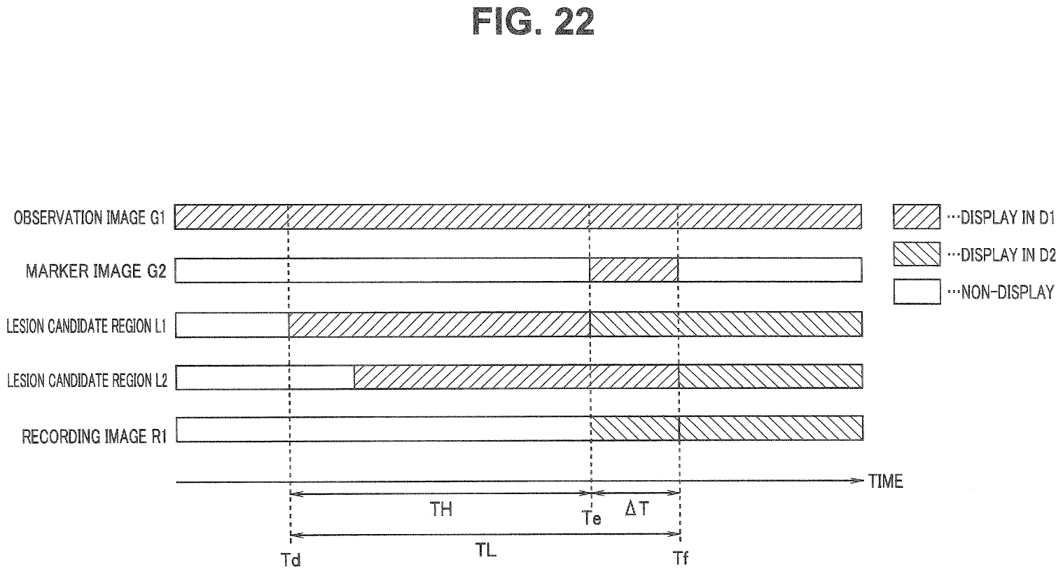

A case where the display control unit 36 performs processing corresponding to a timing chart illustrated in FIG. 22 will be described as an example. FIG. 22 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Td illustrated in FIG. 22, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of the time Td illustrated in FIG. 22, for example

The recording unit 36b in the display control unit 36 starts processing for recording each of the observation images G1 to be sequentially outputted from the video processor 31 as a record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is started at the timing of the time Td illustrated in FIG. 22, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image RE

The highlighting processing unit 36a in the display control unit 36 starts highlighting processing for adding the marker image G2 for highlighting respective positions of the lesion candidate regions L1 and L2 detected by the lesion candidate detection unit 34b to the observation image G1 at a timing at which the predetermined time period TH has elapsed since the consecutive detection time period TL started to be measured based on a determination result to be outputted from the consecutive detection determination unit 35. Note that the detection of the lesion candidate region L1 has ceased at a timing of a time Te illustrated in FIG. 22. Therefore, the marker image G2 is added only to the remaining lesion candidate region L2.

The recording unit 36b in the display control unit 36 stops processing for recording each of the observation images G1 to be sequentially outputted from the video processor 31 as the record image R1 while stopping processing for recording the record image R1 and the lesion candidate information IL to be outputted from the lesion candidate detection unit 34b in association with each other at a timing at which the lesion candidate detection unit 34b has ceased detecting either one of the lesion candidate regions L1 and L2 after the predetermined time period TH has elapsed since the consecutive detection time period TL started to be measured. According to such an operation of the recording unit 36b, processing for recording the record image R1 and the lesion candidate information IL in association with each other is stopped at the timing of the time Te illustrated in FIG. 22, for example. According to the operation of the recording unit 36b described above, the observation images G1 respectively corresponding to X (X≥1) frames are sequentially recorded, respectively, as the record images R1 in a period from the time Td to the time Te illustrated in FIG. 22, for example.

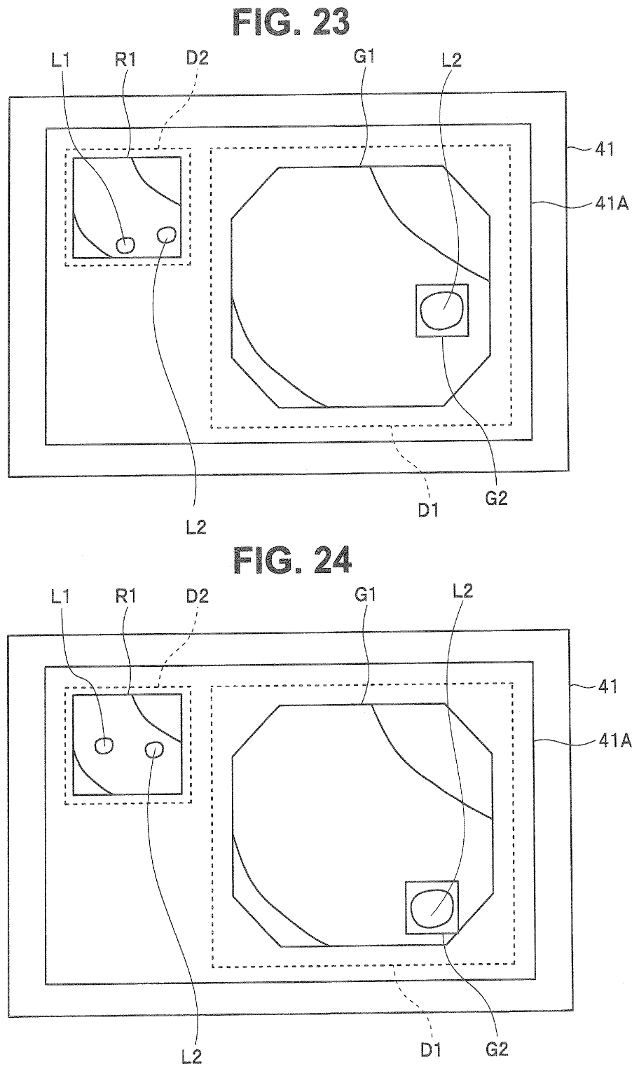

The display control unit 36 starts processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the lesion candidate detection unit 34b has ceased detecting either one of the lesion candidate regions L1 and L2 after the predetermined time period TH has elapsed since the consecutive detection time period TL started to be measured, that is, at the time Te illustrated in FIG. 22. According to such an operation of the display control unit 36, a display image in which the record image R1 is arranged in the display region D2, as illustrated in FIG. 23, is displayed on the display screen 41A in the display apparatus 41 at the timing of the time Te illustrated in FIG. 22, for example. FIG. 23 is a diagram illustrating an example of a display image to be displayed on the display apparatus 41 through the processing of the image processing apparatus 32 according to the embodiment.

The display control unit 36 performs processing for sequentially displaying the record images R1 in an order opposite to an order in which the recording unit 36b has recorded the record images R1 at and after a timing at which the lesion candidate detection unit 34b has ceased detecting the lesion candidate region L1. According to such an operation of the display control unit 36, the record images R1 respectively corresponding to the X frames sequentially recorded by the recording unit 36b, i.e., the X-th frame, the (X−1)-th frame, . . . , the second frame, and the first frame are displayed in this order in the display region D2, for example.

On the other hand, according to an operation of the highlighting processing unit 36a, a rectangular frame surrounding a periphery of the lesion candidate region L2 is added as the marker image G2 to the observation image G1 to be displayed in the display region D1 on the display screen 41A at and after the timing of the time Te illustrated in FIG. 22, which corresponds to a timing at which the predetermined time period TH has elapsed since the lesion candidate detection unit 34b started to detect the lesion candidate region L1, for example. In other words, the observation image G1 in which the marker image G2 is added to the lesion candidate region L2 is displayed in the display region D1 on the display screen 41A in a period from the time Te illustrated in FIG. 22 to a time Tf at which the detection of the lesion candidate region L2 has ceased. At a timing of the time Tf illustrated in FIG. 22, for example, a display image in which the observation image G1 including the lesion candidate region L2 to which the marker image G2 is added is arranged in the display region D1 and the record image R1 is arranged in the display region D2 is displayed on the display screen 41A in the display apparatus 41, as illustrated in FIG. 24. FIG. 24 is a diagram illustrating an example of the display image to be displayed on the display apparatus 41 through the processing of the image processing apparatus 32 according to the embodiment.

In other words, according to the operation of the display control unit 36 as described above, if the consecutive detection time period TL is the predetermined time period TH or more, processing for displaying the plurality of record images R1 recorded during the predetermined time period TH in the display region D2 on the display screen 41A in an order opposite to the recording order by the recording unit 36b while sequentially displaying the plurality of observation images G1 to be sequentially outputted from the video processor 31 in the display region D1 on the display screen 41A is started at the timing of the time Te illustrated in FIG. 22.

Accordingly, even when the consecutive detection time period TL is the predetermined time period TH or more and when the detection of the lesion candidate region L1 ceases before the consecutive detection time period TL, the record image R1 including the lesion candidate regions L1 and L2, which have already been detected by the lesion candidate detection unit 34b, is displayed in the display region D2 on the display screen 41A from a timing at which the detection of the lesion candidate region L1 has ceased so that overlooking of a lesion portion, which can occur in endoscope observation, can be reduced.

Note that a timing at which processing for recording each of the observation images G1 to be sequentially outputted from the video processor 31 as the record image R1 in the recording unit 36b is stopped may be a timing at which the consecutive detection time period TL finishes being measured or a timing at which the detection of all the lesion candidate regions L1 and L2 existing within the observation image G1 ceases. A timing at which the record image R1 recorded by the recording unit 36b starts to be displayed may also be a timing at which the consecutive detection time period TL finishes being measured or a timing at which the detection of all the lesion candidate regions L1 and L2 existing within the observation image G1 ceases.

(d-3) A case where a lesion candidate region L1 is detected before a lesion candidate region L2 and the detection of the lesion candidate region L2 ceases before the detection of the lesion candidate region L1 and a case where a consecutive detection time period TL is less than a predetermined time period TH:

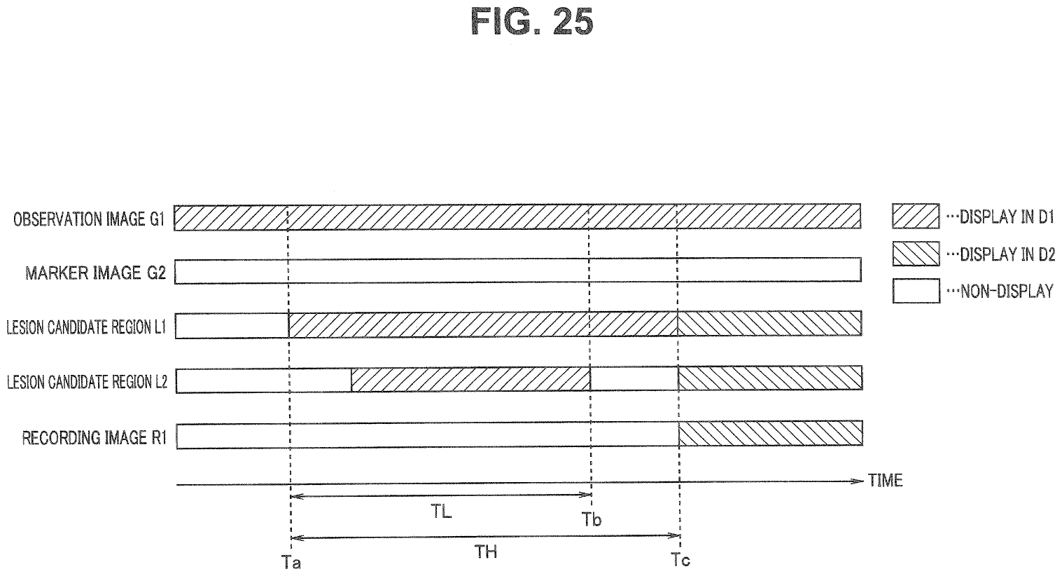

A case where the display control unit 36 performs processing corresponding to a timing chart illustrated in FIG. 25 will be described as an example. FIG. 25 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Ta illustrated in FIG. 25, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of the time Ta illustrated in FIG. 25, for example.

The recording unit 36b in the display control unit 36 starts processing for recording each of the observation images G1 to be sequentially outputted from the video processor 31 as a record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is stated at the timing of the time Ta illustrated in FIG. 25, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image R1.

The recording unit 36b in the display control unit 36 stops processing for recording each of the observation images G1 to be sequentially outputted from the video processor 31 as the record image R1 while stopping processing for recording the record image R1 and the lesion candidate information IL1 and IL2 to be outputted from the lesion candidate detection unit 34b in association with each other at a timing at which the consecutive detection time period TL has finished being measured. For example, processing for recording the record image R1 and the lesion candidate information IL1 and IL2 in association with each other is stopped at a timing of a time Tb illustrated in FIG. 25, for example. According to the operation of the recording unit 36b as described above, the observation images G1 respectively corresponding to Y (Y 1) frames are sequentially recorded, respectively, as the record images R1 in the consecutive detection time period TL corresponding to a period from the time Ta to the time Tb illustrated in FIG. 25, for example.

The display control unit 36 also performs processing for displaying a display image in which the observation image G1 including the lesion candidate region L1 is arranged in the display region D1 on the display screen 41A until the predetermined time period TH has elapsed since the lesion candidate detection unit 34b started to detect the lesion candidate region L1 from a timing at which the consecutive detection time period TL has stopped being measured, that is, a timing at which the lesion candidate detection unit 34b has ceased detecting the lesion candidate region L2. According to such an operation of the display control unit 36, a display image as illustrated in FIG. 19 is displayed on the display screen 41A in the display apparatus 41 at any timing between the time Tb to a time Tc illustrated in FIG. 25, for example.

The display control unit 36 starts, when the detection of the lesion candidate region L2 has ceased before the consecutive detection time period TL reaches the predetermined time period TH based on a determination result to be outputted from the consecutive detection determination unit 35, processing for displaying a display image in which the observation image G1 is arranged in the display region D1 on the display screen 41A and the record image R1 recorded by the recording unit 36b during a measurement period of the predetermined time period TH is arranged in a display region D2 on the display screen 41A at a timing at which the predetermined time period TH has elapsed since the lesion candidate detection unit 34b started to detect the lesion candidate region L1. According to such an operation of the display control unit 36, a display image as illustrated in FIG. 7 is displayed on the display screen 41A in the display apparatus 41 at a timing of the time Tc illustrated in FIG. 25, which corresponds to a timing at which the predetermined time period TH has elapsed since the lesion candidate detection unit 34b started to detect the lesion candidate region L1, for example.

The display control unit 36 performs processing for sequentially displaying the record images R1 in an order opposite to an order in which the recording unit 36b has recorded the record images R1 at and after a timing at which the predetermined time period TH has elapsed since the lesion candidate detection unit 34b started to detect the lesion candidate region L1. According to such an operation of the display control unit 36, the record images R1 respectively corresponding to the Y frames sequentially recorded by the recording unit 36b, i.e., the Y-th frame, the (Y−1)-th frame, . . . , the second frame, and the first frame are displayed in this order in the display region D2, for example.

Accordingly, there may occur a situation where the lesion candidate regions L1 and L2 move outward from inside the observation image G1 with a marker image G2 not displayed in the observation image G1. Accordingly, the lesion candidate region L2 can conceivably be more easily overlooked due to user's viewing.

On the other hand, the record images R1 are displayed in the display region D2 in an order opposite to the recording order by the recording unit 36b. Therefore, respective positions of the lesion candidate regions L1 and L2, which have moved outward from inside the observation image G1, can be notified to a user without the marker image G2 being added to each of the lesion candidate regions L1 and L2, for example.

Further, the lesion candidate region L1 also continues to be displayed in the display region D1 until the predetermined time period TH has elapsed since the lesion candidate region L1 started to be detected even after the detection of the lesion candidate region L2 has ceased. Therefore, the overlooking of the lesion candidate region L1 due to user's viewing can be further reduced. At the timing of the time Tc illustrated in FIG. 25, the lesion candidate region L2 is displayed at a position where the detection of the lesion candidate region L2 has ceased in the display region D2. Therefore, the overlooking of the lesion candidate region L2 can be further reduced.

(d-4) A case where a lesion candidate region L1 is detected before a lesion candidate region L2 and the detection of the lesion candidate region L2 ceases before the detection of the lesion candidate region L1 and a case where a consecutive detection time period TL is a predetermined time period TH or more:

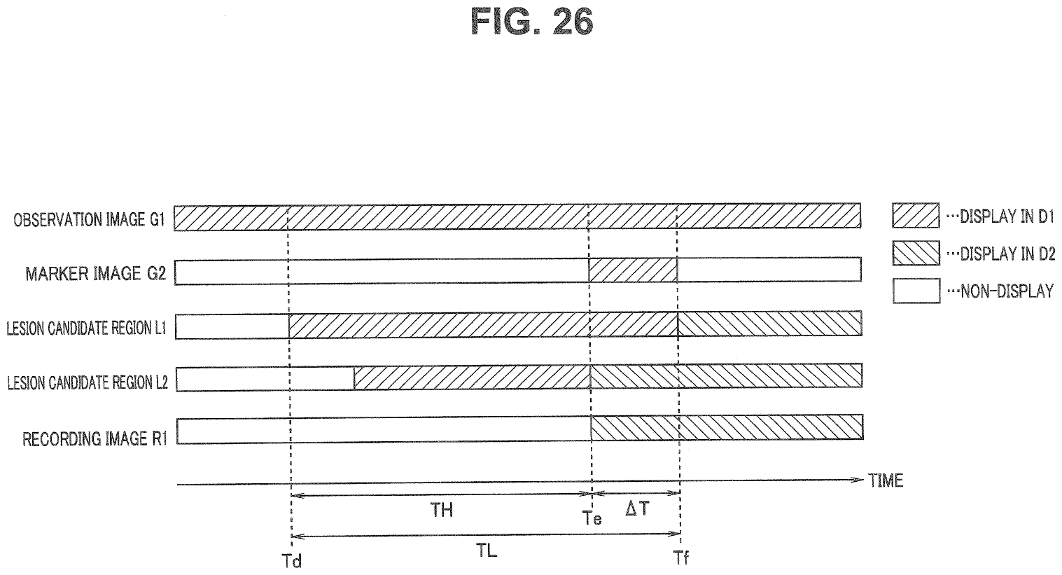

A case where the display control unit 36 performs processing corresponding to a timing chart illustrated in FIG. 26 will be described as an example. FIG. 26 is a timing chart for describing an example different from the example illustrated in FIG. 3 of processing to be performed in the image processing apparatus 32 according to the embodiment.

The display control unit 36 performs processing for displaying a display image in which an observation image G1 is arranged in a display region D1 on the display screen 41A in a period during which the region-of-interest detection unit 34 has not detected the lesion candidate regions L1 and L2. According to such an operation of the display control unit 36, a display image as illustrated in FIG. 4 is displayed on the display screen 41A in the display apparatus 41 in a period before a time Td illustrated in FIG. 26, for example.

The display control unit 36 performs processing for displaying a display image in which the observation image G1 including the lesion candidate region L1 is arranged in the display region D1 on the display screen 41A at a timing at which the consecutive detection time period TL has started to be measured, that is, a timing at which the lesion candidate detection unit 34b has started to detect the lesion candidate region L1. According to such an operation of the display control unit 36, a display image as illustrated in FIG. 19 is displayed on the display screen 41A in the display apparatus 41 at a timing of the time Td illustrated in FIG. 26, for example

The recording unit 36b in the display control unit 36 starts processing for recording each of the observation images G1 to be sequentially outputted from the video processor 31 as the record image R1 while starting processing for recording the record image R1 and the lesion candidate information IL1 to be outputted from the lesion candidate detection unit 34b in association with each other at a timing at which the consecutive detection time period TL has started to be measured. According to such an operation of the recording unit 36b, processing for recording the record image R1 and the lesion candidate information IL1 in association with each other is started at the timing of the time Td illustrated in FIG. 26, for example.

Note that the observation image G1 including the lesion candidate regions L1 and L2, as illustrated in FIG. 5, for example, is displayed in the display region D1 on the display screen 41A from a time point where the lesion candidate detection unit 34b has started to detect the lesion candidate region L2. From the time point, the recording unit 36b in the display control unit 36 also records the lesion candidate information IL2, together with the lesion candidate information IL1 to be outputted from the lesion candidate detection unit 34b, in association with the record image RE

The highlighting processing unit 36a in the display control unit 36 starts highlighting processing for adding a marker image G2 for highlighting respective positions of the lesion candidate regions L1 and L2 detected by the lesion candidate detection unit 34b to the observation image G1 at a timing at which the predetermined time period TH has elapsed since the consecutive detection time period TL started to be measured based on a determination result to be outputted from the consecutive detection determination unit 35. Note that at a timing of a time Te illustrated in FIG. 26, the detection of the lesion candidate region L2 has ceased. Therefore, the marker image G2 is added only to the remaining lesion candidate region L1.

The recording unit 36b in the display control unit 36 stops processing for recording each of the observation images G1 to be sequentially outputted from the video processor 31 as the record image R1 while stopping processing for recording the record image R1 and the lesion candidate information IL to be outputted from the lesion candidate detection unit 34b in association with each other at a timing at which the lesion candidate detection unit 34b has ceased detecting either one of the lesion candidate regions L1 and L2 after the predetermined time period TH has elapsed since the consecutive detection time period TL started to be measured. According to such an operation of the recording unit 36b, processing for recording the record image R1 and the lesion candidate information IL in association with each other is stopped at the timing of the time Te illustrated in FIG. 26, for example. According to the operation of the recording unit 36b as described above, the observation images G1 respectively corresponding to Z (Z 1) frames are sequentially recorded, respectively, as the record images R1 in a period from the time Td to the time Te illustrated in FIG. 26, for example.

The display control unit 36 starts processing for displaying a display image in which the observation image G1 is arranged in the display region D1 on the display screen 41A and the record image R1 recorded by the recording unit 36b during a measurement period of the consecutive detection time period TL is arranged in a display region D2 on the display screen 41A at a timing at which the lesion candidate detection unit 34b has ceased detecting either one of the lesion candidate regions L1 and L2, that is, at the time Te illustrated in FIG. 26 after the predetermined time period TH has elapsed since the consecutive detection time period TL started to be measured. According to such an operation of the display control unit 36, a display image in which the record image R1 is arranged in a display region D2, as illustrated in FIG. 16, is displayed on the display screen 41A in the display apparatus 41 at the timing of the time Te illustrated in FIG. 26, for example.

The display control unit 36 performs processing for sequentially displaying the record images R1 in an order opposite to an order in which the recording unit 36b has recorded the record images R1 at and after a timing at which the lesion candidate detection unit 34b has ceased detecting the lesion candidate region L2. According to such an operation of the display control unit 36, the record images R1 respectively corresponding to the Z frames sequentially recorded by the recording unit 36b, i.e., the Z-th frame, the (Z−1)-th frame, . . . , the second frame, and the first frame are displayed in this order in the display region D2, for example.

On the other hand, according to an operation of the highlighting processing unit 36a, a rectangular frame surrounding a periphery of the lesion candidate region L1 is added as the marker image G2 to the observation image G1 to be displayed in the display region D1 on the display screen 41A at and after the timing of the time Te illustrated in FIG. 26, which corresponds to a timing at which the predetermined time period TH has elapsed since the lesion candidate detection unit 34b started to detect the lesion candidate region L1, for example. In other words, the observation image G1 in which the marker image G2 is added to the lesion candidate region L1 is displayed in the display region D1 on the display screen 41A in a period from the time Te in FIG. 26 to a time Tf at which the detection of the lesion candidate region L1 ceases. At a timing of the time Tf illustrated in FIG. 26, for example, a display image in which the observation image G1 including the lesion candidate region L2 to which the marker image G2 is added is arranged in the display region D1 and the record image R1 is arranged in the display region D2, as illustrated in FIG. 17, is displayed on the display screen 41A in the display apparatus 41.

In other words, according to the operation of the display control unit 36 as described above, if the consecutive detection time period TL is the predetermined time period TH or more, processing for displaying the plurality of record images R1 recorded during the predetermined time period TH in the display region D2 on the display screen 41A in an order opposite to the recording order by the recording unit 36b while sequentially displaying the plurality of observation images G1 to be sequentially outputted from the video processor 31 in the display region D1 on the display screen 41A at the timing of the time Te illustrated in FIG. 26.

Accordingly, even when the consecutive detection time period TL is the predetermined time period TH or more and when the detection of the lesion candidate region L2 ceases before the consecutive detection time period TL, the record image R1 including the lesion candidate regions L1 and L2, which have already been detected by the lesion candidate detection unit 34b, is displayed in the display region D2 on the display screen 41A from a timing at which the detection of the lesion candidate region L2 has ceased so that overlooking of a lesion portion, which can occur in endoscope observation, can be reduced.

Note that a timing at which processing for recording each of the observation images G1 to be sequentially outputted from the video processor 31 as the record image R1 in the recording unit 36b is stopped may be a timing at which the consecutive detection time period TL finishes being measured or a timing at which the detection of all the lesion candidate regions L1 and L2 existing within the observation image G1 ceases. A timing at which the record image R1 recorded by the recording unit 36b starts to be displayed may also be a timing at which the consecutive detection time period TL finishes being measured or a timing at which the detection of all the lesion candidate regions L1 and L2 existing within the observation image G1 ceases.

Note that in any one of the above-described 10 patterns (a-1) to (d-4), the processing for displaying the record images R1 in the display region D2 in the order opposite to the recording order by the recording unit 36b need not necessarily be performed in the display control unit 36. For example, processing for displaying the record image R1 in the display region D2 in the same order as the recording order by the recording unit 36b may be performed in the display control unit 36.

The processing for sequentially displaying in the display region D2 the record images R1 respectively corresponding to the frames recorded during the measurement period of the consecutive detection time period TL need not necessarily be performed in the display control unit 36. For example, processing for displaying in the display region D2 some of the record images R1 respectively corresponding to the frames may be performed in the display control unit 36. More specifically, processing for displaying in the display region D2 only the record image R1 corresponding to the one frame finally recorded among the record images R1 respectively corresponding to the frames recorded during the measurement period of the consecutive detection time period Tl may be performed in the display control unit 36, for example. Alternatively, processing for displaying the plurality of record images R1 recorded during the measurement period of the consecutive detection time period TL in the display region D2 while decimating the record images R1 at a predetermined spacing may be performed in the display control unit 36.

For example, the recording unit 36b may record each of the observation images G1 obtained by decimating the plurality of observation images G1 to be sequentially outputted from the video processor 31 during the measurement period of the consecutive detection time period TL at a predetermined spacing as the record image R1.

The recording unit 36b need not necessarily sequentially record the observation images G1 corresponding to a plurality of frames, respectively, as the record images RE For example, the recording unit 36b may record only the observation image corresponding to one frame as the record image RE More specifically, the recording unit 36b may record only the observation image G1 inputted to the display control unit 36 at a timing immediately before the consecutive detection time period TL stops being measured as the record image R1, for example.

When the display control unit 36 displays in the display region D2 the record image R1 corresponding to each of the frames recorded in the recording unit 36b, the record image R1 may be displayed at constant speed at the same frame rate as a frame rate at the time of recording by the recording unit 36b, may be displayed at double speed at a higher frame rate than the frame rate at the time of recording by the recording unit 36b, or may be displayed slow at a lower frame rate than the frame rate at the time of recording by the recording unit 36b.

When the display control unit 36 displays in the display region D2 the record image R1 recorded in the recording unit 36b, the record image R1 may be displayed in the same color as a color at the time of the recording by the recording unit 36b, may be displayed with a reduced number of colors as compared with the colors at the time of the recording by the recording unit 36b, or may be displayed in only one predetermined color.

As long as the record image R1 is recorded during the measurement period of the consecutive detection time period TL, the record image R1 may start to be recorded from a desired timing before the timing at which the lesion candidate region L1 starts to be detected, for example.

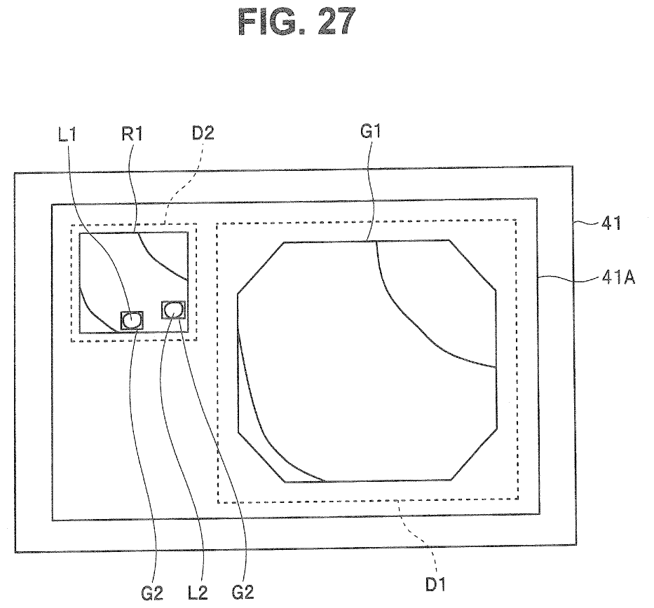

When the display control unit 36 displays in the display region D2 the record image R1 recorded in the recording unit 36b, the highlighting processing unit 36a may perform highlighting processing for generating the marker image G2 based on the lesion candidate information IL1 and IL2 recorded with each of the lesion candidate information IL1 and IL2 associated with the record image R1 and adding the generated marker image G2 to the record image R1, for example. According to such an operation of the display control unit 36, a display image in which the respective positions of the lesion candidate regions L1 and L2 included in the record image R1 in the display region D2 are highlighted, as illustrated in FIG. 27, can be displayed on the display screen 41A at the timing of the time Tc illustrated in FIG. 3 or the timing of the time Tf illustrated in FIG. 8, for example. FIG. 27 is a diagram illustrating an example of a display image to be displayed on the display apparatus 41 through the processing of the image processing apparatus 32 according to the embodiment.

The operation of the display control unit 36 as described above is also applied in substantially the same manner when a record image R1 including three or more lesion candidate regions is recorded and displayed (including highlighting processing for adding a marker image G2).

The present invention is not limited to the above-described embodiment, but it goes without saying that various modifications and applications are possible without departing from the scope and spirit of the invention.

The image processing apparatus and the like according to the present embodiment may include a processor and a storage (e.g., a memory). The functions of individual units in the processor may be implemented respective pieces of hardware or may be implemented by an integrated piece of hardware, for example. The processor may include hardware, and the hardware may include at least one of a circuit for processing digital signals and a circuit for processing analog signals, for example. The processor may include one or a plurality of circuit devices (e.g., an IC) or one or a plurality of circuit elements (e.g., a resistor, a capacitor) on a circuit board, for example. The processor may be a CPU (central processing unit), for example, but this should not be construed in a limiting sense, and various types of processors including a GPU (graphics processing unit) and a DSP (digital signal processor) may be used. The processor may be a hardware circuit with an ASIC (application specific integrated circuit) or an FPGA (field-programmable gate array). The processor may include an amplification circuit, a filter circuit, or the like for processing analog signals. The memory may be a semiconductor memory such as an SRAM and a DRAM; a register; a magnetic storage device such as a hard disk device; and an optical storage device such as an optical disk device. The memory stores computer-readable instructions, for example. When the instructions are executed by the processor, the functions of each unit of the image processing device and the like are implemented. The instructions may be a set of instructions constituting a program or an instruction for causing am operation on the hardware circuit of the processor.

The units in the image processing apparatus and the like and the display apparatus according to the present embodiment may be connected with each other via any types of digital data communication such as a communication network or via communication media. The communication network may include a LAN (local area network), a WAN (wide area network), and computers and networks which form the internet, for example.

What is claimed is:

1. An endoscope image processing apparatus including a processor, wherein the processor
performs processing for sequentially receiving a plurality of observation images acquired by performing image pickup of an object and respectively detecting regions of interest for the plurality of observation images,
sequentially records the plurality of observation images as record images in either one of a first period from a first detection start at which a first region of interest starts to be detected to a first detection cessation at which detection of the first region of interest ceases and a second period from the first detection start to a second detection cessation at which detection of a second region of interest ceases when the second region of interest is detected in the first period,
calculates a display timing at which the plurality of record images start to be reproduced based on a time point of at least one of the first detection start, the first detection cessation, a second detection start at which the second region of interest starts to be detected, and the second detection cessation, and
performs processing for displaying at least one of the recorded record images on a display screen of a display apparatus while sequentially displaying the plurality of observation images on the display screen at the display timing.

2. The image processing apparatus according to claim 1, wherein
the processor selects as the first detection start either one of a detection start of a region of interest detected earliest among the regions of interest and a detection start of a region of interest highest in likelihood of a polyp among the regions of interest.

3. The image processing apparatus according to claim 1, wherein
the processor selects at least one of the detection starts and selects at least one of the detection cessations, to determine whether or not the recorded record image is displayed on the display screen and determine whether or not at least one of the regions of interest is highlighted and is displayed on the display screen at a timing at which a predetermined time period elapses from the selected detection start or at a time point of the selected detection cessation.

4. The image processing apparatus according to claim 1, wherein
the processor displays information indicating that the regions of interest are detected in a second display region while sequentially displaying the plurality of observation images in a first display region on the display screen.

5. The image processing apparatus according to claim 1, wherein
the processor performs processing for displaying one or more of the recorded record images in a second display region on the display screen while sequentially displaying the plurality of observation images in a first display region on the display screen.

6. The image processing apparatus according to claim 5, wherein
the second display region is set as a region having a smaller size than a size of the first display region on the display screen.

7. The image processing apparatus according to claim 1, wherein
the processor detects the regions of interest based on a feature value of a pixel in an acquired image of the object.

8. The image processing apparatus according to claim 1, wherein
the processor performs processing for sequentially displaying the recorded record images on the display screen in an order opposite to a recording order of the recorded record images.

9. The image processing apparatus according to claim 1, wherein
the processor performs processing for displaying the recorded record images on the display screen while decimating the record images at a predetermined spacing.

10. The image processing apparatus according to claim 1, wherein
the processor records each of the observation images obtained by decimating the plurality of observation images at a predetermined spacing as a record image in the period.

11. The image processing apparatus according to claim 5, wherein
the processor respectively records the plurality of observation images acquired before a timing of the detection cessation as one or more of record images in an acquired order, and
decimates and displays in the second display region the plurality of record images.

12. A non-transitory storage medium storing a computer readable program for causing a computer to perform
processing for respectively detecting regions of interest for a plurality of observation images acquired by performing image pickup of an object,
processing for sequentially recording the plurality of observation images as record images in either one of a first period from a first detection start at which a first region of interest starts to be detected to a first detection cessation at which detection of the first region of interest ceases and a second period from the first detection start to a second detection cessation at which detection of a second region of interest ceases when the second region of interest is detected in the first period, and
processing for calculating a display timing at which the plurality of record images start to be reproduced based on a time point of at least one of the first detection start, the first detection cessation, a second detection start at which the second region of interest starts to be detected, and the second detection cessation.

13. The non-transitory storage medium according to claim 12, wherein
the storage medium stores a program for causing a computer to perform processing for selecting either one of a detection start of a region of interest earliest detected among the regions of interest and a detection start of a region of interest highest in likelihood of a polyp among the regions of interest as the first detection start.

14. The non-transitory storage medium according to claim 12, wherein
the storage medium stores a program for causing a computer to perform processing for selecting at least one of the detection starts and selecting at least one of the detection cessations, to determine whether or not the record image is displayed on a display screen and determine whether or not at least one of the regions of interest is highlighted and is displayed on the display screen at a timing at which a predetermined time period elapses after the selected detection start or at a time point of the selected detection cessation.

15. The non-transitory storage medium according to claim 12, wherein
the storage medium stores a program for causing a computer to perform processing for displaying at least one of the record images in a second display region on a display screen while sequentially displaying the plurality of observation images in a first display region on the display screen.
